(19) United States
(12) Patent Application Publication
(10) Pub. No.: US 2014/0200184 A1
(43) Pub. Date: Jul. 17, 2014

(54) Chitinase-3-Like Protein 1 as a Biomarker of Recovery from Kidney Injury

(71) Applicant: YALE UNIVERSITY, New Haven, CT (US)

(72) Inventors: Jack A. Elias, Woodbridge, CT (US); Chirag R. Parikh, Wallingford, CT (US); Lloyd G. Cantley, East Haven, CT (US)

(73) Assignee: YALE UNIVERSITY, New Haven, CT (US)

(21) Appl. No.: 14/166,264

(22) Filed: Jan. 28, 2014

Related U.S. Application Data
(63) Continuation of application No. 13/474,011, filed on May 17, 2012.
(60) Provisional application No. 61/487,076, filed on May 17, 2011.

(51) Int. Cl. G01N 33/68; A61K 38/47; C12Q 1/68

(52) U.S. Cl. CPC G01N 33/6893; C12Q 1/6883; A61K 38/47
USPC 514/15.4; 436/501; 435/7.92; 435/6.11; 435/6.12; 506/9; 435/7.1

(57) ABSTRACT

The present invention provides compositions and methods for the detection, treatment, and prevention of a kidney injury. The invention relates to the discovery that chitinase 3-like 1/Brp-39/YKL-40 serves as both a biomarker for the degree of kidney injury and a critical mediator of a reparative response in the kidney.

### CROSS-REFERENCE TO RELATED APPLICATIONS

This application is a continuation of and claims priority to U.S. application Ser. No. 13/474,011, filed May 17, 2012, which claims priority under 35 U.S.C. §119(e) to U.S. provisional application No. 61/487,076, filed May 17, 2011, each of which application is hereby incorporated herein by reference in its entirety.

### STATEMENT REGARDING FEDERALLY SPONSORED RESEARCH OR DEVELOPMENT

This invention was made with government support under HL108638 awarded by National Institute of Health (NIH). The government has certain rights in the invention.

### BACKGROUND OF THE INVENTION

Chitinase-like family of secreted proteins (CLPs) are evolutionarily conserved 18 glycosyl hydrolase proteins that bind but do not cleave chitin (Elias et al., 2005, J Allergy Clin Immunol 116:497-500). The best studied CLP is chitinase 3-like 1 (CHI3L1) that encodes the human protein YKL-40 and the mouse orthologue Brp-39. Recent studies have demonstrated that these moieties are important regulators of innate and adaptive immunity, tissue injury, apoptosis, TGF-β1 elaboration and parenchymal scarring (Rejman and Hurley, 1988, Biochem Biophys Res Commun 150:329-334; Hakala et al., 1993, J Biol Chem 268:25803-25810; Lee et al., 2009, The Journal of Experimental Medicine 206: 1149-1166; Hard et al., 2009, Journal of Immunology 182:5098-5106). YKL-40 is produced by a variety of cells including neutrophils, monocytes, macrophages, chondrocytes, synovial cells, smooth muscle cells, endothelial and tumor cells (Johansen et al., 1995, Eur J Cancer 31A:1437-1442; Ober, and Chupp, 2009, Curr Opin Allergy Clin Immunol 9:401-408; Hakala et al., 1993, J Biol Chem 268:25803-25810) and is readily detected in the blood of normal individuals (Bojesen et al., 2011, Clin Chim Acta 412:709-712). Elevated circulating levels of YKL-40 have been observed in patients with asthma, metastatic breast cancer, cardiovascular disease, type 2 diabetes and hepatic fibrosis (Chupp et al., 2007, N Engl J Med 357:2016-2027; Shackel et al., 2003, Hepatology 38:577-588; Rathcke and Vestergaard, 2009, Cardiovasc Diabetol 8:61). In many of these disorders YKL-40 correlates with disease activity and its expression is believed to reflect distinct pathways in disease pathogenesis (Fontana et al., 2010, Gut 59:1401-1409; Thorn et al., 2010, Cancer 116:4114-4121; Francescone et al., 2011, J Biol Chem. 286(17):15332-43).

Renal ischemia (RI), occurring in clinical settings such as sepsis, cardiopulmonary bypass or kidney transplantation, can lead to acute kidney injury (AKI) that in turn triggers complex pathophysiologic responses associated with increased morbidity, mortality and hospital length of stay (Molitoris et al., 2007, Nat Clin Pract Nephrol 3:439-442; Coca et al., 2009, Am J Kidney Dis 53:961-973; Wald et al., 2009, JAMA 302:1179-1185; James et al., 2010, Lancet 376:2096-2103). Unfortunately no successful therapeutic interventions that either limit initial kidney injury or accelerate the subsequent repair has been established with current treatment focused on mechanical replacement of kidney filtration function in anticipation of successful regeneration of the damaged nephrons via endogenous repair mechanisms. For this reason, morbidity and mortality rates in patients who develop AKI have remained stable or improved only slightly over the past several decades, while the incidence of AKI has steadily increased (Waikar et al., 2006, J Am Soc Nephrol 17:1143-1150; Anderson et al., 2011, J Am Soc Nephrol 22:28-38).

While most studies of AKI have focused on the initiation of injury and its associated risk factors and outcomes, there are far fewer studies that address the biology and clinical patterns of recovery. There is an urgent need in the art for new therapeutic targets for the treatment of AKI. The present invention fills this need.

### SUMMARY OF THE INVENTION

The present invention provides a method of identifying a subject as having a kidney injury. In one embodiment, the method comprises the steps of: a) measuring the level of YKL-40 present in a first body sample obtained from a first subject; b) measuring the level of YKL-40 present in a second body sample obtained from a second subject not having a kidney injury; c) comparing the level of YKL-40 in the first body sample obtained from the first subject to the level of YKL-40 present in a second body sample obtained from a second subject not having a kidney injury; wherein, when the level of YKL-40 is elevated in the first body sample compared to the level of YKL-40 present in the second body sample, the first subject is identified as having a kidney injury.

The invention also provides a method of predicting recovery from an acute kidney injury in a subject. In one embodiment, the method comprising the steps of: a) measuring the level of YKL-40 present in a first body sample obtained from a first subject at a first time point; b) measuring the level of YKL-40 present in a second body sample obtained from the subject at a later time point; c) comparing the level of YKL-40 in the first body sample to the level of YKL-40 present in the second body sample; wherein, when the level of YKL-40 is elevated in the second body sample compared to the level of YKL-40 present in the first body sample, the subject is predicted to be recovering from an acute kidney injury.

The invention also provides a method of predicting delayed graft function after kidney transplantation in a subject. In one embodiment, method comprises the steps of: a) measuring the level of YKL-40 present in a first body sample obtained from a first subject at a first time point; b) measuring the level of YKL-40 present in a second body sample obtained from the subject at a later time point; c) comparing the level of YKL-40 in the first body sample to the level of YKL-40 present in the second body sample; wherein, when the level of YKL-40 is elevated in the second body sample compared to the level of YKL-40 present in the first body sample, the delayed graft function after kidney transplantation is predicted in the subject.

The invention provides a method of diagnosing for dialysis treatment in a subject following kidney transplantation. In one embodiment, the method comprises the steps of: a) measuring the level of YKL-40 present in a first body sample obtained from a first subject at a first time point; b) measuring the level of YKL-40 present in a second body sample obtained from the subject at a later time point; c) comparing the level of YKL-40 in the first body sample to the level of YKL-40 present in the second body sample; wherein, when the level of YKL-40 is elevated in the second body sample compared to the level of YKL-40 present in the first body sample, the subject is diagnosed for needing dialysis treatment.

The invention provides a method of treating a subject diagnosed with a kidney injury. In one embodiment, the method comprises administering to the subject a composition comprising a therapeutically effective amount of YKL-40 or an activator of YKL-40 wherein the composition attenuates, prevents, or halts kidney cell apoptosis.

In one embodiment, the subject is a mammal, preferably, the mammal is a human.

In one embodiment, the body sample is at least one body sample selected from the group consisting of a tissue, a cell, and a body fluid.

In one embodiment, the body fluid is urine.

In one embodiment, the measuring of the YKL-40 comprises an immunoassay for assessing the level of the YKL-40 in the sample.

In one embodiment, the immunoassay is at least one immunoassay selected from the group consisting of Western blot, ELISA, immunoprecipitation, immunohistochemistry, immunofluorescence, radioimmunoassay, dot blotting, and FACS.

In one embodiment, the measuring of the YKL-40 comprises a nucleic acid assay for assessing the level of a nucleic acid encoding the YKL-40 in the sample.

In one embodiment, the nucleic acid assay is at least one nucleic acid assay selected from the group consisting of a Northern blot, Southern blot, in situ hybridization, a PCR assay, an RT-PCR assay, a probe array, and a gene chip.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1D, is a series of images demonstrating that Chi3l1/Brp-39 is upregulated following ischemic kidney injury. FIG. 1A is an image depicting the results of an experiment where urine was obtained on the indicated days from 5 male mice subjected to 25 minutes of bilateral renal ischemia, pooled, and immunoblotted using α-Brp-39. FIG. 1B is an image depicting the results of an experiment where RNA was harvested on the indicated days from kidneys subjected to 25 minutes of ischemia and quantitative RT-PCR performed for Chi3l1 (normalized to Hprt, n=4 kidneys at each time point). FIG. 1C is an image depicting the results of an experiment where urine was obtained 48 hours after sham operation, 15 minutes or 35 minutes of FR was pooled from 3 male mice and immunoblotted using α-mouse Chi311 (R&D Systems). FIG. 1D is an image depicting the results of an experiment where qRT-PCR for Chi3l1 was performed on kidney RNA harvested 48 hours after sham, 15 or 35 minutes of I/R. n=5, *p<0.01 vs. control. Creatinine values are shown for the three groups at 48 hours after injury. n=3 or 4 for each group, **p<0.01 vs. 15 minutes or control.

FIG. 2, comprising FIGS. 2A through 2E, is a series of images depicting the results of experiments demonstrating that Brp-39 is required for the normal repair phase after acute kidney injury. FIG. 2A is an image depicting Kaplan-Meier survival analysis of male WT or Brp-39^{-/-} mice subjected to 30 minutes of unilateral I/R with contralateral nephrectomy and followed for 7 days. n=21 for WT and 19 for Brp-39^{-/-}, p=0.001. FIGS. 2B and 2C are images depicting creatinine and BUN values obtained at the indicated times from mice subjected to 25 minutes I/R that survived until day 3. For creatinine, n=7 for each group, p=ns on day 1 and p<0.05 on day 3. For BUN, n=10 WT and 11 Brp39^{-/-} mice, p<0.05 for Brp39^{-/-} vs. control at 1 and 3 days. FIG. 2D is an image depicting H&E staining from representative regions of the outer medulla of WT and Brp-39^{-/-} mice 3 days after 25 minutes of I/R. FIG. 2E is an image depicting Tissue Injury Scoring performed on kidney sections from mice treated as in FIG. 2D and reported as % of tubules with cells exhibiting overt necrosis (n=3 on day 1, n=4 on day 3. *p=0.01 vs. WT).

FIG. 3, comprising FIGS. 3A through 3G, is a series of images depicting the results of experiments demonstrating that Brp-39 protects against tubular cell apoptosis via activation of PI3-Kinase. FIG. 3A is representative images of outer medulla from kidney sections obtained from WT and Brp39^{-/-} mice 3 days after 25 minutes of I/R and stained for TUNEL+ apoptotic cells (staining indicating TUNEL+ nuclei, magnification 400×). FIG. 3B is an image depicting the quantification of apoptotic tubular cells identified as in FIG. 3A (n=5, *p<0.01). FIG. 3C is representative images of outer medulla from kidney sections obtained as in FIG. 3A and stained for Ki-67+ proliferating cells (staining indicating Ki-67+ nuclei, magnification 400×). FIG. 3D is an image depicting quantification of proliferating tubular cells identified as in FIG. 3C (n=5, *p<0.01). FIG. 3E is an image demonstrating that cultured PTEC were serum starved overnight and stimulated for the indicated time with recombinant Brp-39 (10 μM), followed by lysis and immunoblotting with pAkt and total Akt (lower panel). Upper panel shows quantification of pAkt from 3 separate experiments normalized to tAkt. *p<0.05 vs. baseline. FIG. 3F is an image depicting cells treated as in FIG. 3E and immunoblotted for pErk (p42/44) and total Erk. n=3, *p<0.05 vs. 0 or 1 hour. FIG. 3G is an image depicting that PTEC were treated for 6 hours with H₂O₂±Brp-39±LY294002 and then fixed and stained for apoptosis using TUNEL. n=4 separate experiments, *p<0.05 vs. control; **p<0.05 vs. H₂O₂ alone; ***p=ns vs. H₂O₂ alone.

FIG. 4, comprising FIGS. 4A and 4B, is a series of images demonstrating that urinary and serum YKL-40 levels are elevated in patients with DGF. Urinary and blood YKL-40 values measured at different time points by level of allograft recovery (mean±SEM). FIG. 4A is an image depicting urinary YKL-40 concentrations at the indicated times after transplantation. p<0.001 for DGF vs. either SGF or IGF. p=ns for SGF vs. IGF. FIG. 4B is an image depicting blood YKL-40 levels at the indicated times. Day 0 samples were obtained immediately after transplant.

FIG. 5 depicts receiver-operating characteristics curves for predicting DGF using urine and blood YKL-40 at 0 hours and the first POD with associated area under the curve ±SEM.

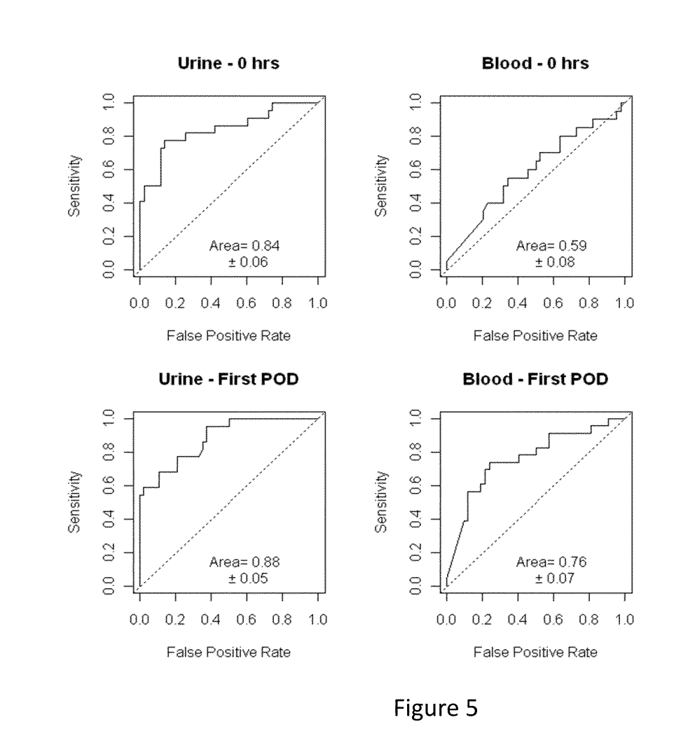

FIG. 6 is an image depicting Kaplan-Meier survival analysis of male WT or Brp-39^{-/-} mice subjected to 25 minutes of unilateral I/R with contralateral nephrectomy and followed for 3 days. N=24 for each group.

FIG. 7 depicts a summary of the FACS profile of macrophages isolated from WT and Brp-39^{-/-} kidneys.

FIG. 8 depicts the summary of baseline and clinical characteristics of transplant recipients and donors. Values are ±SD or N (percent of total). DGF, delayed graft function defined by dialysis within one week of transplant; SGF, slow graft function defined by <70% reduction in serum creatinine by day seven without need for dialysis; IFG, immediate graft function defined by absence of SGF without need for dialysis, ECD, extended-criteria donor; DCD, donation after cardiac death; ESRD, end-stage renal disease; PRA, panel reactive antibody; HLA, human leukocyte antigen; Scr, serum creatinine.

FIG. 9 depicts mean and median YKL-40 results by level of allograft function after transplant.

FIG. 10 depicts the results of an analysis evaluating the accuracy of urine and blood YKL-40 for predicting DGF.

FIG. 11 depicts the results of an analysis evaluating the sensitivity, specificity and likelihood ratios for predicting DGF using selected urine and blood YKL-40 cutoff values. Optimal cutoff identified as the value with the largest sum of sensitivity plus specificity. LR+, likelihood ratio for a positive test; LR−, likelihood ratio for a negative test.

### DETAILED DESCRIPTION OF THE INVENTION

The present invention includes compositions and methods for diagnosing and treating a kidney disease in a subject.

The present invention provides a biomarker for diagnosing and treating kidney injury in a subject. The biomarkers of the invention are also useful for the assessment of the severity of kidney injury in a subject. The biomarker of the invention includes but is not limited to chitinase-like family of secreted proteins (CLPs). Preferably, the CLP is chitinase 3-like 1/Brp-39/YKL-40.

In one embodiment, the present invention provides a method for detecting, diagnosing, prognosing, or monitoring the risk of acute kidney injury (AKI) by measuring the levels of a CLP, preferably chitinase 3-like 1/Brp-39/YKL-40, in a subject.

In one embodiment, the biomarkers of the invention can be used for predicting recovery of AKI in a subject.

In one embodiment, the biomarker of the invention can be used to rapidly identify a subject at risk of having sustained renal failure following transplantation. In some instances, urinary levels of chitinase 3-like 1/Brp-39/YKL-40 obtained within hours of transplant are highly predictive of the need for subsequent dialysis in the subject, wherein higher levels of chitinase 3-like 1/Brp-39/YKL-40 in the urine is predictive for the need of dialysis.

In another embodiment, the biomarker of the invention can be used for early recognition of rejection of kidney transplantation, wherein the presence of the marker of the invention is indicative of the existence of rejection after kidney transplantation. In some instances, the biomarker of the invention can be used to distinguish between subjects who have delayed graft function following ischemia/reperfusion (I/R) injury compared to those who have immediate graft function.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The phrase “activator,” as used herein, means to enhance a molecule, a reaction, an interaction, a gene, an mRNA, and/or a protein's expression, stability, function or activity by a measurable amount.

The term “antibody,” as used herein, refers to an immunoglobulin molecule that is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By the term “applicator,” as the term is used herein, is meant any device including, but not limited to, a hypodermic syringe, a pipette, and the like, for administering the compounds and compositions of the invention.

The terms “biomarker” and “marker” are used herein interchangeably. They refer to a substance that is a distinctive indicator of a biological process, biological event and/or pathologic condition.

The phrase “body sample” or “biological sample” is used herein in its broadest sense. A sample may be of any biological tissue or fluid from which biomarkers of the present invention may be assayed. Examples of such samples include but are not limited to blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological or body samples may also include sections of tissues such as frozen sections taken for histological purposes. The sample also encompasses any material derived by processing a biological or body sample. Derived materials include, but are not limited to, cells (or their progeny) isolated from the sample, proteins or nucleic acid molecules extracted from the sample. Processing of a biological or body sample may involve one or more of: filtration, distillation, extraction, concentration, inactivation of interfering components, addition of reagents, and the like.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

In the context of the present invention, the term “control,” when used to characterize a subject, refers, by way of non-limiting examples, to a subject that is healthy, to a patient that has been diagnosed with a renal disease (e.g., end-stage chronic kidney disease), or to a renal transplant patient that has been diagnosed with a stable renal transplant, with renal transplant glomerulopathy or with interstitial fibrosis and tubular atrophy (IFTA). The term “control sample” refers to one, or more than one, sample that has been obtained from a healthy subject or from a patient diagnosed with a particular kidney status or renal transplant status.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold higher or more, as compared with a control.

“Differentially decreased expression” or “down regulation” refers to biomarker product levels which are at least 10% or more, for example, 20%, 30%, 40%, or 50%, 60%, 70%, 80%, 90% lower or less, and/or 0.9 fold, 0.8 fold, 0.6 fold, 0.4 fold, 0.2 fold, 0.1 fold or less, as compared with a control.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

Signal transduction is any process by which a cell converts one signal or stimulus into another, most often involving ordered sequences of biochemical reactions carried out within the cell. The number of proteins and molecules participating in these events increases as the process emanates from the initial stimulus resulting in a “signal cascade.” The phrase “downstream effector,” as used herein, refers to a protein or molecule acted upon during a signaling cascade, which in term acts upon another protein or molecule. The term “downstream” indicates the direction of the signaling cascade.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

The term “dysregulation” as used herein describes an over- or under-expression of chitinase 3-like 1/Brp-39/YKL-40 in an individual with renal failure as compared to a normal individual without renal failure.

As used herein “endogenous” refers to any material from or produced inside the organism, cell, tissue or system.

As used herein, the term “exogenous” refers to any material introduced from or produced outside the organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

As used herein, the terms “indicative of kidney status” and “indicative of renal transplant status,” when applied to a process or event, refers to a process or event which is indicative of a kidney status or a renal transplant status, such that the process or event is found significantly more often in subjects with a given kidney status or a given renal transplant status than in subjects with a different kidney status or a different renal transplant status (as determined using routine statistical methods). Preferably, a protein biomarker which is indicative of a given kidney status (e.g., end-stage kidney disease) or a given renal transplant status (e.g., stable renal transplant, renal transplant glomerulopathy or IFTA) is recognized by at least 60% of subjects who exhibit the kidney status or the renal transplant status, respectively and is recognized by less than 10% of subjects who do not exhibit the kidney status or the renal transplant status. More preferably, a protein biomarker which is indicative of a given kidney status or of a given renal transplant status is recognized by at least 70%, at least 75%, at least 80%, at least 85%, at least 90%, at least 95% or more patients who exhibit the same kidney status or the same renal transplant status and is recognized by less than 10%, less than 8%, less than 5%, less than 2% or less than 1% subjects who do not exhibit the same kidney status or the same renal transplant status.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The “level” of one or more biomarkers means the absolute or relative amount or concentration of the biomarker in the sample.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “normal” and “healthy” are used herein interchangeably. They include an individual or group of individuals who have not undergone kidney transplantation and who have not shown any signs or symptoms of kidney injury, damage or dysfunction. The term “normal” is also used herein to qualify a sample (e.g., a blood sample) obtained from a healthy individual.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

By the term “specifically binds,” as used herein, is meant a molecule, such as an antibody, which recognizes and binds to another molecule or feature, but does not substantially recognize or bind other molecules or features in a sample.

As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “subject” refers to a human or another mammal (e.g., primate, dog, cat, goat, horse, pig, mouse, rat, rabbit, and the like) that can undergo kidney transplantation, but may or may not have undergone kidney transplantation. In many embodiments of the present invention, the subject is a human being. In such embodiments, the subject is often referred to as an “individual” or a “patient.” As used herein, the term “kidney transplant patient” refers to an individual that has undergone kidney transplantation. The terms “individual” and “patient” do not denote a particular age.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based on the discovery of the role of chitinase 3-like 1/Brp-39/YKL-40 in the kidney, its roles in the pathogenesis of acute or other forms of kidney injury and its utility as a biomarker for renal diseases. The invention is also based on the unexpected discovery of the role of chitinase-like proteins in ischemic organ injury and the concept that the innate immune response has evolved to identify and dispose of cells that are severely injured while promoting the survival and expansion of sublethally injured cells to affect subsequent organ repair.

The discovery of chitinase 3-like 1/Brp-39/YKL-40 as both a sensor of the degree of kidney injury and a critical mediator of the reparative response in the kidney provides a powerful biomarker that can be used to rapidly identify patients that are at greatest risk to have sustained renal failure following transplantation. Accordingly, the invention provides compositions and methods useful in identifying an individual with a renal disease, or an individual at risk of developing a renal disease. In one aspect of the invention, the individual is a mammal. In a preferred aspect of the invention, the mammal is a human.

### Biomarker

A biomarker is an organic biomolecule which is differentially present in a sample taken from an individual of one phenotypic status (e.g., having a disease) as compared with an individual of another phenotypic status (e.g., not having the disease). A biomarker is differentially present between the two individuals if the mean or median expression level of the biomarker in the different individuals is calculated to be statistically significant. Biomarkers, alone or in combination, provide measures of relative risk that an individual belongs to one phenotypic status or another. Therefore, they are useful as markers for diagnosis of disease, the severity of disease, therapeutic effectiveness of a drug, and drug toxicity.

Accordingly, the invention provides methods for identifying one or more biomarkers which can be used to aid in the diagnosis, detection, and prediction of renal disease, such as a kidney disorder. The methods of the invention are carried out by obtaining a set of measured values for a plurality of biomarkers from a biological sample derived from a test individual, obtaining a set of measured values for a plurality of biomarkers from a biological sample derived from a control individual, comparing the measured values for each biomarker between the test and control sample, and identifying biomarkers which are significantly different between the test value and the control value, also referred to as a reference value.

The process of comparing a measured value and a reference value can be carried out in any convenient manner appropriate to the type of measured value and reference value for the biomarker of the invention. For example, “measuring” can be performed using quantitative or qualitative measurement techniques, and the mode of comparing a measured value and a reference value can vary depending on the measurement technology employed. For example, when a qualitative calorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the invention will most commonly be quantitative values (e.g., quantitative measurements of concentration). In other examples, measured values are qualitative. As with qualitative measurements, the comparison can be made by inspecting the numerical data, or by inspecting representations of the data (e.g., inspecting graphical representations such as bar or line graphs).

A measured value is generally considered to be substantially equal to or greater than a reference value if it is at least about 95% of the value of the reference value. A measured value is considered less than a reference value if the measured value is less than about 95% of the reference value. A measured value is considered more than a reference value if the measured value is at least more than about 5% greater than the reference value.

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a desired biomarker. Alternately, a separate device (e.g., a digital computer) may be used to compare the measured value(s) and the reference value(s). Automated devices for comparison may include stored reference values for the biomarker(s) being measured, or they may compare the measured value(s) with reference values that are derived from contemporaneously measured reference samples.

### Diagnostic

The invention relates in part on the discovery that levels of urinary chitinase-like family of secreted proteins (CLPs) are markedly increased after kidney injury, correlating with upregulated renal expression of the mRNA for Chi3l1 that peaks during the time of kidney repair. In addition, the level of renal Chi3l1 mRNA expression and urinary Brp-39 (YKL-40) excretion directly correlates with the severity of kidney injury.

The present invention also relates partly to the discovery that upregulation of chitinase 3-like 1/Brp-39/YKL-40 in response to ischemic injury is important in inhibiting tubular cell apoptosis in vivo and that the chitinase 3-like 1/Brp-39/YKL-40 pathway serves to limit the severity of tubular injury and maintain sufficient kidney function to keep the subject alive and promote proliferation of viable tubular cells to effect subsequent kidney repair.

Accordingly, the present invention relates generally to diagnostic methods and markers, prognostic methods and markers, and therapy evaluators for kidney disorders, such as acute kidney injury (AKI).

In certain embodiments, the method comprises the step of obtaining a sample of urine from the subject, and assessing the level of chitinase 3-like 1/Brp-39/YKL-40 in the urine sample. Thus, the present invention relates to markers for determining renal transplant or kidney status in a subject, methods for diagnosis of a kidney disorder, methods of determining predisposition to a kidney disorder, methods of monitoring progression/regression of a kidney disorder, methods of assessing efficacy of compositions for treating a kidney disorder, methods of screening compositions for activity in modulating markers of a kidney disorder, methods of treating a kidney disorder, as well as other methods based on markers of a kidney disorder.

In certain embodiments, the invention further provides methods for permitting refinement of disease diagnosis, disease risk prediction, and clinical management of individuals associated with a kidney disorder. The markers of the invention represent a urine-based assay for assessing a kidney disorder that can be used for determining the disease state or disease risk. The detection of the selective markers of the invention in a subject, or a sample obtained therefrom, permits refinement of disease diagnosis, disease risk prediction, and clinical management of individuals being treated with agents that are associated with a kidney disorder.

In one embodiment, the biomarker can also be used to predict recovery from kidney injury in a subject. In another embodiment, the biomarker can be used to predict graft function after kidney transplantation. In another embodiment, the biomarker can be used for predicting delayed graft function. In yet another embodiment, the biomarker of the invention can be used to determine whether a transplant recipient requires subsequent dialysis. Also provided are methods, arrays and kits for using the biomarkers of the invention for determining renal transplant or kidney status in a subject.

In one embodiment of the invention provides a method for identifying an individual with a kidney injury comprising detecting or measuring chitinase 3-like 1/Brp-39/YKL-40 in a body sample obtained from an individual diagnosed with a kidney injury, or a putative at-risk individual, then comparing the levels of chitinase 3-like 1/Brp-39/YKL-40 present in the test sample to chitinase 3-like 1/Brp-39/YKL-40 levels detected or measured in a sample obtained from one or more otherwise identical, normal, not-at-risk individuals. In some instances, the level of chitinase 3-like 1/Brp-39/YKL-40 expression is compared with an average value obtained from more than one not-at-risk individual. In other instances, the level of chitinase 3-like 1/Brp-39/YKL-40 expression is compared with chitinase 3-like 1/Brp-39/YKL-40 assessed in a sample obtained from one normal, not-at-risk individual. In yet another instance, the level of chitinase 3-like 1/Brp-39/YKL-40 expression in the putative at-risk individual is compared with the level of chitinase 3-like 1/Brp-39/YKL-40 expression in a sample obtained from the same individual at a different time.

In another embodiment, a method of diagnosing a kidney disorder in a subject comprises the steps of obtaining a first sample of urine from the subject at a first time; assessing the level of a marker of the invention in the first urine sample to obtain a baseline level; obtaining a second sample of urine from the subject at a second time and assessing the level of the marker in the second urine sample to obtain a second level. If the second level is significantly higher compared to the baseline level, the subject is at an increased risk of developing or having a kidney disorder. In one embodiment, the second level is also compared to a reference population of a subject without the kidney disorder; if the second level is significantly higher compared to the level derived from a reference population, the subject is at an increased risk of developing or having a kidney disorder.

One aspect of the present invention provides a biomarker to detect a kidney disorder. In another aspect, the invention provides a biomarker to determine renal transplant or kidney status in a subject, methods for diagnosis of a kidney disorder, methods of determining predisposition for developing a kidney disorder, methods of monitoring progression/regression of a kidney disorder, methods of assessing efficacy of compositions for treating a kidney disorder, methods of screening compositions for activity in modulating markers of a kidney disorder, methods of treating a kidney disorder, as well as other methods based on markers of a kidney disorder.

A biomarker is typically a protein, found in a bodily fluid, whose level varies with disease state and may be readily quantified. The quantified level may then be compared to a known value. The comparison may be used for several different purposes, including but not limited to, diagnosis of a kidney disorder, prognosis of a kidney disorder, and monitoring treatment of a kidney disorder.

In some embodiments, the invention provides a kit with a detection reagent which binds to chitinase 3-like 1/Brp-39/YKL-40, fragments, analogs, metabolites, or other analytes.

In some embodiments, a detection reagent is immobilized on a solid matrix such as a porous strip or bead to form at least one kidney injury biomarker detection site.

The levels of a biomarker of the invention may be assessed in several different biological samples, for example bodily fluids. Non-limiting examples of bodily fluid include whole blood, plasma, serum, bile, lymph, pleural fluid, semen, saliva, sweat, urine, and cerebral spinal fluid. In one embodiment, the bodily fluid is selected from the group of whole blood, plasma, and serum. In another embodiment, the bodily fluid is whole blood. In yet another embodiment, the bodily fluid is plasma. In still yet another embodiment, the bodily fluid is serum. Preferably, the bodily fluid is urine.

The bodily fluid is obtained from the individual using conventional methods in the art. For instance, one skilled in the art knows how to draw blood and how to process it in order to obtain serum and/or plasma for use in the method. Generally speaking, the method preferably maintains the integrity of the biomarkers of the invention such that it can be accurately quantified in the bodily fluid. Methods for collecting blood or fractions thereof are well known in the art. For example, see U.S. Pat. No. 5,286,262, which is hereby incorporated by reference in its entirety.

In a preferred embodiment, the sample is a urine sample or blood sample, and the blood sample may be a (blood) serum or (blood) plasma sample.

Urine samples can be taken as known in the prior art. Preferably, a midstream urine sample is used in the context of the present disclosure. For example, the urine sample may be taken by means of a catheter or also by means of a urination apparatus as described in WO 01/74275.

A bodily fluid may be obtained from any mammal known or suspected to suffer from a kidney disorder or that can be used as a disease model for a kidney disorder. In one embodiment, the mammal is a rodent. Examples of rodents include mice, rats, and guinea pigs. In another embodiment, the mammal is a primate. Examples of primates include monkeys, apes, and humans. In an exemplary embodiment, the mammal is a human. In some embodiments, the individual has no clinical signs of a kidney disorder. In other embodiments, the individual has mild clinical signs of a kidney disorder. In yet other embodiments, the individual may be at risk for a kidney disorder. In still other embodiments, the individual has been diagnosed with a kidney disorder.

Assessment of biomarker levels may encompass assessment of the level of protein concentration or the level of enzymatic activity of the biomarker, wherever applies. In either case, the level is quantified such that a value, an average value, or a range of values is determined. In one embodiment, the level of protein concentration of the kidney disorder biomarker is quantified.

There are numerous known methods and kits for measuring the amount or concentration of a protein in a sample, including as non-limiting examples, ELISA, western blot, absorption measurement, colorimethc determination, Lowry assay, Bicinchoninic acid assay, or a Bradford assay. Commercial kits include ProteoQwest™ Colohmetric Western Blotting Kits (Sigma-Aldrich, Co.), QuantiPro™ bicinchoninic acid (BCA) Protein Assay Kit (Sigma-Aldrich, Co.), FluoroProfile™ Protein Quantification Kit (Sigma-Aldrich, Co.), the Coomassie Plus—The Better Bradford Assay (Pierce Biotechnology, Inc.), and the Modified Lowry Protein Assay Kit (Pierce Biotechnology, Inc.). In certain embodiments, the protein concentration is measured using a luminex based multiplex immunoassay panel. However, the invention should not be limited to any particular assay for assessing the level of a biomarker of the invention. That is, any currently known assay used to detect protein levels and assays to be discovered in the future can be used to detect the biomarkers of the invention.

Methods of quantitatively assessing the level of a protein in a biological sample such as plasma are well known in the art. In some embodiments, assessing the level of a protein involves the use of a detector molecule for the biomarker. Detector molecules can be obtained from commercial vendors or can be prepared using conventional methods available in the art. Exemplary detector molecules include, but are not limited to, an antibody that binds specifically to the biomarker, a naturally-occurring cognate receptor, or functional domain thereof, for the biomarker, or a small molecule that binds specifically to the biomarker.

In a preferred embodiment, the level of a biomarker is assessed using an antibody. Thus, non-limiting exemplary methods for assessing the level of a biomarker in a biological sample include various immunoassays, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY.

Other methods for assessing the level of a protein include chromatography (e.g., HPLC, gas chromatography, liquid chromatography) and mass spectrometry (e.g., MS, MS-MS). For instance, a chromatography medium comprising a cognate receptor for the biomarker or a small molecule that binds to the biomarker can be used to substantially isolate the biomarker from the biological sample. Small molecules that bind specifically to a biomarker can be identified using conventional methods in the art, for instance, screening of compounds using combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection.

The level of a substantially isolated protein can be quantitated directly or indirectly using a conventional technique in the art such as spectrometry, Bradford protein assay, Lowry protein assay, biuret protein assay, or bicinchoninic acid protein assay, as well as immunodetection methods.

In another embodiment, the level of enzymatic activity of the biomarker if such biomarker has an enzymatic activity may be quantified. Generally, enzyme activity may be measured by means known in the art, such as measurement of product formation, substrate degradation, or substrate concentration, at a selected point(s) or time(s) in the enzymatic reaction. There are numerous known methods and kits for measuring enzyme activity. For example, see U.S. Pat. No. 5,654,152. Some methods may require purification of the biomarker prior to measuring the enzymatic activity of the biomarker. A pure biomarker constitutes at least about 90%, preferably, 95% and even more preferably, at least about 99% by weight of the total protein in a given sample. Biomarkers of the invention may be purified according to methods known in the art, including, but not limited to, ion-exchange chromatography, size-exclusion chromatography, affinity chromatography, differential solubility, differential centrifugation, and HPLC.

As apparent from the examples disclosed herein, diagnostic tests that use the biomarkers of the invention exhibit a sensitivity and specificity of at least 75%, at least 80%, at least 85%, at least 90%, at least 95%, at least 98% and about 100%. In some instances, screening tools of the present invention exhibits a high sensitivity of at least 75%, at least 80%, at least 85%, at least 90%, at least 95%, at least 98% and about 100%. Without wishing to be bound by any particular theory, it is believed that screening tools should exhibit high sensitivity, but specificity can be low. However, diagnostics should have high sensitivity and specificity.

Determining kidney disease status typically involves classifying an individual into one of two or more groups based on the results of the diagnostic test. The diagnostic tests described herein can be used to classify an individual into a number of different states. In one embodiment, the invention provides methods for determining the presence or absence of a kidney disease in an individual (status: kidney disease v. non-kidney disease). The presence or absence of kidney disease is determined by measuring the relevant biomarker or biomarkers in samples obtained from individuals and then either submitting them to a classification algorithm or comparing them with a reference amount and/or pattern of biomarkers that is associated with the particular risk level.

In another embodiment, the invention provides methods for determining the risk of developing disease in an individual. Biomarker amounts or patterns are characteristic of various risk states, e.g., high, medium or low. The risk of developing kidney disease is determined by measuring the relevant biomarker or biomarkers in sample obtained from individuals and then either submitting them to a classification algorithm or comparing them with a reference amount and/or pattern of biomarkers that is associated with the particular risk level.

In yet another embodiment, the invention provides methods for determining the stage of kidney disease in an individual. Each stage of the disease can be characterized by the amount of a biomarker or relative amounts of a set of biomarkers (i.e., a pattern) that are found in a sample obtained from the individual. The stage of kidney disease is determined by measuring the relevant biomarker or biomarkers and then either submitting them to a classification algorithm or comparing them with a reference amount and/or pattern of biomarkers that is associated with the particular stage.

In another embodiment, the invention provides methods for determining the course of kidney disease in an individual. Disease course refers to changes in disease status over time, including disease progression (worsening) and disease regression (improvement). Over time, the amounts or relative amounts (e.g., the pattern) of the biomarkers changes. For example, levels of various biomarkers of the present invention increase with progression of disease. Accordingly, this method involves measuring the level of one or more biomarkers in an individual at two or more different time points, e.g., a first time and a second time, and comparing the change in amounts. The course of disease is determined based on these comparisons.

In some instances, the levels of various biomarkers of the invention decreases with disease progression. In this method, the level of one or more biomarkers in a sample from an individual is measured at two or more different time points, e.g., a first time and a second time, and the change in levels, if any is assessed. The course of disease is determined based on these comparisons.

Similarly, changes in the rate of disease progression (or regression) may be monitored by measuring the level of one or more biomarkers at different times and calculating the rate of change in biomarker levels. The ability to measure disease state or rate of disease progression is important for drug treatment studies where the goal is to slow down or arrest disease progression using therapy.

Additional embodiments of the invention relate to the communication of the results or diagnoses or both to technicians, physicians or patients, for example. In certain embodiments, computers are used to communicate results or diagnoses or both to interested parties, e.g., physicians and their patients.

In certain embodiments, the methods of the invention further comprise managing individual treatment based upon their disease status. Such management includes the actions of the physician or clinician subsequent to determining kidney disease status. For example, if a physician makes a diagnosis of a kidney disease, then a certain regime of treatment, such as prescription or administration of the therapeutic compound might follow. Alternatively, a diagnosis of a non-kidney disease might be followed by further testing to determine any other diseases that might the patient might be suffering from. Also, if the test is inconclusive with respect to a kidney disease status, further tests may be called for.

In a preferred embodiment of the invention, a diagnosis based on the presence or absence or relative levels in the biological sample of an individual of the relevant biomarkers disclosed herein is communicated to the individual as soon as possible after the diagnosis is obtained.

According to yet another aspect, the present invention provides a method of assessing efficacy of a treatment of a kidney disease in a patient comprising: a) determining a baseline level of biomarkers in a first sample obtained from the patient before receiving the treatment; b) determining the level of same biomarkers in a second sample obtained from the patient after receiving the treatment; wherein an increase in the levels of the biomarkers in the post-treatment sample is correlated with a positive treatment outcome.

### Detection Methods

Any methods available in the art for identification or detection of chitinase 3-like 1/Brp-39/YKL-40 are encompassed herein. Chitinase 3-like 1/Brp-39/YKL-40 can be detected at a nucleic acid level or a protein level. In order to determine up-regulation of chitinase 3-like 1/Brp-39/YKL-40 expression, levels of the chitinase 3-like 1/Brp-39/YKL-40 are measured in the body sample to be examined and compared with a corresponding body sample that originates from a normal, not-at-risk individual. In another embodiment of the invention, up-regulation of chitinase 3-like 1/Brp-39/YKL-40 is determined by measuring levels of chitinase 3-like 1/Brp-39/YKL-40 in the body sample to be examined and comparing with an average value obtained from more than one not-at-risk individual. In still another embodiment of the invention, up-regulation of chitinase 3-like 1/Brp-39/YKL-40 is determined by measuring levels of chitinase 3-like 1/Brp-39/YKL-40 in the body sample to be examined and comparing with levels of chitinase 3-like 1/Brp-39/YKL-40 obtained from a body sample obtained from the same individual at a different time.

Methods for detecting chitinase 3-like 1/Brp-39/YKL-40 comprise any method that determines the quantity or the presence of chitinase 3-like 1/Brp-39/YKL-40 either at the nucleic acid or protein level. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. In particular embodiments, chitinase 3-like 1/Brp-39/YKL-40 is detected on a protein level using, for example, antibodies that are directed against chitinase 3-like 1/Brp-39/YKL-40 protein. These antibodies can be used in various methods such as Western blot, ELISA, immunoprecipitation, or immunocytochemistry techniques.

The invention should not be limited to any one method of protein or nucleic acid detection method recited herein, but rather should encompass all known or heretofore unknown methods of detection as are, or become, known in the art.

Samples may need to be modified in order to render the chitinase 3-like 1/Brp-39/YKL-40 antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a chitinase 3-like 1/Brp-39/YKL-40 is then incubated with the sample.

Techniques for detecting antibody binding are well known in the art. Antibody binding to chitinase 3-like 1/Brp-39/YKL-40 may be detected through the use of chemical reagents that generate a detectable signal that corresponds to the level of antibody binding and, accordingly, to the level of chitinase 3-like 1/Brp-39/YKL-40 protein expression. In one of the preferred immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil., incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for chitinase 3-like 1/Brp-39/YKL-40 protein. Methods for making antibodies and for selecting appropriate antibodies are known in the art. See, for example, Celis, J. E. ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the chitinase 3-like 1/Brp-39/YKL-40 protein, and method of body sample preparation. Furthermore, the detection chemistry used to visualize antibody binding to a chitinase 3-like 1/Brp-39/YKL-40 protein must also be optimized to produce the desired signal to noise ratio.

Immunoassays

Immunoassays, in their simplest and most direct sense, are binding assays. Certain preferred immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the chitinase 3-like 1/Brp-39/YKL-40 proteins of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the chitinase 3-like 1/Brp-39/YKL-40 antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the chitinase 3-like 1/Brp-39/YKL-40, and detected by means of their label. The amount of marker antigen in an unknown sample is then determined by mixing the sample with the labeled antibodies before or during incubation with coated wells. The presence of chitinase 3-like 1/Brp-39/YKL-40 antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immune complex can be detected with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

Nucleic Acid-Based Techniques

In other embodiments, the expression of chitinase 3-like 1/Brp-39/YKL-40 is detected at the nucleic acid level. Nucleic acid-based techniques for assessing expression are well known in the art and include, for example, determining the level of chitinase 3-like 1/Brp-39/YKL-40 mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target biomolecule, for example, a nucleotide transcript or a protein encoded by or corresponding to chitinase 3-like 1/Brp-39/YKL-40. Probes can be synthesized by one of skill in the art, or derived from appropriate biological preparations. Probes may be specifically designed to be labeled with a detectable label. Examples of molecules that can be used as probes include, but are not limited to, RNA, DNA, proteins, antibodies, and organic molecules.

Isolated mRNA can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding chitinase 3-like 1/Brp-39/YKL-40 of the present invention. Hybridization of an mRNA with the probe indicates that the chitinase 3-like 1/Brp-39/YKL-40 in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for determining the level of chitinase 3-like 1/Brp-39/YKL-40 mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self-sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), and rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan™ System). Such methods typically use pairs of oligonucleotide primers that are specific for the biomarker of interest. Methods for designing oligonucleotide primers specific for a known sequence are well known in the art.

Chitinase 3-like 1/Brp-39/YKL-40 expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Treatment

The present invention is based on the discovery that of the factors identified by a proteomic analysis of mouse urine after ischemia/reperfusion (I/R) injury, fragments of the chitinase-like family of secreted proteins (CLPs) were observed to be most highly upregulated in the urine during kidney repair.

The disclosure presented herein demonstrates that upregulation of Brp-39 in response to ischemic injury is critical in inhibiting tubular cell apoptosis in vivo, and that this pathway serves to limit the severity of tubular injury and maintain sufficient kidney function to keep the mammal alive and promote proliferation of viable tubular cells to effect subsequent kidney repair. Mice lacking Brp-39 demonstrate significantly worse outcomes following ischemia/reperfusion (I/R) compared to control animals, with more severe tubular injury and apoptosis, persistent reduction of kidney function and decreased survival. In vitro assays revealed that Brp-39 stimulates intracellular activation of the PI3K/AKT pathway in renal tubular cells, resulting in decreased apoptosis in response to oxygen radical exposure.

It has also been demonstrated herein that BRP-39/YKL-40 serves a biologically relevant role in the ischemically injured kidney. Chi3l1 mRNA is upregulated in an injury-dependent fashion following renal I/R in the mouse, with increased B-39 protein levels in the urine. Similarly, patients who have delayed graft function following ischemia/reperfusion (I/R) injury during kidney transplantation exhibit a marked increase in urinary YKL-40 levels as compared to those who have immediate graft function.

Accordingly, the invention provides compositions and methods useful in treating an individual diagnosed with a kidney disorder (e.g., kidney injury). Treating an individual diagnosed with a kidney disorder encompasses a method of inhibiting the progression of a kidney disorder in an individual diagnosed with a kidney disorder. By “inhibiting the progression of a kidney disorder” is intended to mean that the progressive histological and morphometric changes associated with the clinical sequelae of a kidney disorder, for example cell death is halted, prevented, or attenuated. It will be appreciated that the method of the present invention may also be practiced in an individual at risk of developing a kidney disorder whereby an individual identified as being at risk of developing a kidney disorder may be prevented from developing or experiencing cell death that would subsequently lead to a clinical manifestation of a kidney disorder.

The methods of the invention comprise administering a therapeutically effective amount of a chitinase 3-like 1/Brp-39/YKL-40 or an activator thereof to a subject with a kidney disorder or an individual at risk of developing a kidney disorder where the chitinase 3-like 1/Brp-39/YKL-40 or an activator thereof reduces or prevents, halts, or attenuates cell death.

Enhancing or increasing chitinase 3-like 1/Brp-39/YKL-40 expression or activity can be accomplished using any method known to the skilled artisan. Examples of methods to enhance or increase chitinase 3-like 1/Brp-39/YKL-40 expression include, but are not limited to increasing expression of an endogenous chitinase 3-like 1 gene, increasing expression of chitinase 3-like 1/Brp-39/YKL-40 mRNA, and increasing expression of chitinase 3-like 1/Brp-39/YKL-40 protein. An agent, composition or compound that enhances or increases chitinase 3-like 1/Brp-39/YKL-40 expression or activity may be a compound or composition that increases expression of a chitinase 3-like 1 gene, a compound or composition that increases chitinase 3-like 1/Brp-39/YKL-40 mRNA half-life, stability and/or expression, or a compound or composition that enhances chitinase 3-like 1/Brp-39/YKL-40 protein function. An agent, composition or compound that enhances or increases chitinase 3-like 1/Brp-39/YKL-40 expression or activity may be any type of compound, including but not limited to, a polypeptide, a nucleic acid, an aptamer, a peptidometic, and a small molecule, or combinations thereof.

The present invention should in no way be construed to be limited to the activators described herein, but rather should be construed to encompass any activator of chitinase 3-like 1/Brp-39/YKL-40, both known and unknown, that promotes kidney repair or prevents, attenuates, or halts cell death of a kidney cell such as renal tubular cells.

The treatment methods of the invention comprises administering a therapeutically effective amount of chitinase 3-like 1/Brp-39/YKL-40 or an agent that enhances or increases chitinase 3-like 1/Brp-39/YKL-40 expression or activity to a mammal in need thereof in order to promote repair of the kidney.

In another embodiment, the method of the invention comprises a therapeutically effective amount of chitinase 3-like 1/Brp-39/YKL-40 or an agent that enhances or increases chitinase 3-like 1/Brp-39/YKL-40 expression or activity to a mammal to treat a mammal diagnosed with a disease or disorder wherein dysregulation of chitinase 3-like 1/Brp-39/YKL-40 is a component of the disease or disorder.

The mammal may be diagnosed with a disease or disorder wherein the disease or disorder has a dysregulation of the expression of chitinase 3-like 1/Brp-39/YKL-40 and corresponding pathway in kidney as part of the disease's clinical features. Alternatively, the subject may be at-risk of developing a disease or disorder wherein the disease or disorder has a dysregulation of chitinase 3-like 1/Brp-39/YKL-40 and corresponding pathway in kidney as part of the disease's clinical features.

Methods of prophylaxis (i.e., prevention or decreased risk of disease), as well as reduction in the frequency or severity of symptoms associated with kidney disorder or any related disease or disorder, are encompassed by the present invention.

The method of the invention comprises administering a therapeutically effective amount of chitinase 3-like 1/Brp-39/YKL-40 or an agent that enhances or increases chitinase 3-like 1/Brp-39/YKL-40 expression or activity to a mammal in combination with other therapeutic agents to treat a the mammal A chitinase 3-like 1/Brp-39/YKL-40 or an agent that enhances or increases chitinase 3-like 1/Brp-39/YKL-40 expression or activity may be administered, before, during, after, or throughout the administration of the therapeutic agent. The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone)), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

Isolated nucleic acid-based chitinase 3-like 1/Brp-39/YKL-40 or an agent that enhances or increases chitinase 3-like 1/Brp-39/YKL-40 expression or activity can be delivered to a cell in vitro or in vivo using viral vectors comprising one or more sequences corresponding to chitinase 3-like 1/Brp-39/YKL-40 or an agent that enhances or increases chitinase 3-like 1/Brp-39/YKL-40 expression or activity. Generally, the nucleic acid sequence has been incorporated into the genome of the viral vector. The viral vector comprising an isolated nucleic acid described herein can be contacted with a cell in vitro or in vivo and infection can occur. The cell can then be used experimentally to study, for example, the effect of an isolated sequence in vitro, or the cells can be implanted into a subject for therapeutic use. The cell can be migratory, such as a hematopoietic cell, or non-migratory. The cell can be present in a biological sample obtained from the subject (e.g., blood, bone marrow, tissue, fluids, organs, etc.) and used in the treatment of disease, or can be obtained from cell culture.

After contact with the viral vector comprising an isolated sequence corresponding to chitinase 3-like 1/Brp-39/YKL-40 or an agent that enhances or increases chitinase 3-like 1/Brp-39/YKL-40 expression or activity, the sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is removed from the subject or animal and returned to the subject or animal once contacted with the viral vector comprising the isolated nucleic acid of the present invention. Ex vivo gene therapy has been described, for example, in Kasid et al., Proc. Natl. Acad. Sci. USA 87:473 (1990); Rosenberg et al, New Engl. J. Med. 323:570 (1990); Williams et al., Nature 310476 (1984); Dick et al., Cell 42:71 (1985); Keller et al., Nature 318:149 (1985) and Anderson et al., U.S. Pat. No. 5,399,346 (1994).

Where a cell is contacted in vitro, the cell incorporating the viral vector comprising the desired nucleic acid can be implanted into a subject or experimental animal model for delivery or used in in vitro experimentation to study cellular events mediated by chitinase 3-like 1/Brp-39/YKL-40 or an agent that enhances or increases chitinase 3-like 1/Brp-39/YKL-40 expression or activity.

Various viral vectors can be used to introduce a desired nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver a desired nucleic acid of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, a desired nucleic acid sequence of the invention can be delivered to cells without vectors, e.g. as “naked” nucleic acid delivery using methods known to those of skill in the art. See, for example, U.S. Pat. Nos. 5,350,674 and 5,585,362.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Various forms of a desired nucleic acid sequence of the invention can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated nucleic acid of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of polynucleotides into a cell.

Further, liposomes can be used as carriers to deliver a nucleic acid to a cell, tissue or organ. Liposomes comprising neutral or anionic lipids do not generally fuse with the target cell surface, but are taken up phagocytically, and the polynucleotides are subsequently subjected to the degradative enzymes of the lysosomal compartment (Straubinger et al., 1983, Methods Enzymol. 101:512-527; Mannino et al., 1988, Biotechniques 6:682-690). Methods of delivering a nucleic acid to a cell, tissue or organism, including liposome-mediated delivery, are known in the art and are described in, for example, Felgner (Gene Transfer and Expression Protocols Vol. 7, Murray, E. J. Ed., Humana Press, New Jersey, (1991)).

In other related aspects, the invention includes an isolated nucleic acid sequence corresponding to chitinase 3-like 1/Brp-39/YKL-40 or an agent that enhances or increases chitinase 3-like 1/Brp-39/YKL-40 expression or activity that is operably linked to a nucleic acid comprising a promoter/regulatory sequence. Thus, the invention encompasses expression vectors and methods for the introduction of an isolated chitinase 3-like 1/Brp-39/YKL-40 sequence or a sequence corresponding to an agent that enhances or increases chitinase 3-like 1/Brp-39/YKL-40 expression or activity into cells.

Such delivery can be accomplished by generating a plasmid, viral, or other type of vector comprising an isolated chitinase 3-like 1/Brp-39/YKL-40 sequence or sequence of an activator thereof operably linked to a promoter/regulatory sequence which serves to introduce the chitinase 3-like 1/Brp-39/YKL-40 or activator thereof into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of the desired nucleic acid sequence may be accomplished by placing an isolated chitinase 3-like 1/Brp-39/YKL-40 sequence or a sequence of an activator thereof, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

Any expression vector compatible with the expression of chitinase 3-like 1/Brp-39/YKL-40 or a an activator thereof of the invention is suitable for use in the instant invention, and can included but is not limited to a plasmid DNA, a viral vector, a mammalian vector, and the like. The expression vector, or a vector that is co-introduced with the expression vector, can further comprise a marker gene. Marker genes are useful, for instance, to monitor transfection efficiencies. Marker genes include: genes for selectable markers, including but not limited to, G418, hygromycin, and methotrexate, and genes for detectable markers, including, but not limited to, luciferase and GFP. The expression vector can further comprise an integration signal sequence which facilitates integration of the isolated polynucleotide into the genome of a target cell.

### Pharmaceutical

The therapeutic and prophylactic methods of the invention thus encompass the use of pharmaceutical compositions comprising chitinase 3-like 1/Brp-39/YKL-40 or an activator thereof. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses.

As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

A kit is envisaged for every method described herein. The following description of a kit useful for diagnosing a kidney disorder or disease in a subject by measuring the level of a biomarker of the invention in a biological sample therefore is not intended to be limiting and should not be construed that way. Preferably, the biomarker is chitinase 3-like 1/Brp-39/YKL-40.

The kit may comprise a negative control containing a biomarker at a concentration of about the concentration of the biomarker which is present in a biological sample of an individual who does not have a kidney disorder or disease or does not have increased risk for kidney disorder or disease. The kit may also include a positive control containing the biomarker at a concentration of about the concentration of the biomarker which is present in a biological sample of an individual who as a kidney disorder or disease or has increased risk for a kidney disorder or disease.

All the materials and reagents required for assaying chitinase 3-like 1/Brp-39/YKL-40 according to the present invention can be assembled together in a kit, such kit includes at least elements in aid of assessing the level of chitinase 3-like 1/Brp-39/YKL-40 in a biological sample obtained from an individual, and the instruction on how to do so.

A kit of the invention may have materials and reagents for detecting the chitinase 3-like 1/Brp-39/YKL-40 levels such as an immunoassay, or parts required to perform an immunoassay specific for chitinase 3-like 1/Brp-39/YKL-40 detection. Optionally, a kit may further or alternatively comprise elements for performing PCR based assays for the detection of chitinase 3-like 1/Brp-39/YKL-40 and determination of levels of the same from biological samples. The kit of parts may further comprise equipment for obtaining one or more biological samples, such equipment may for example be syringes, vials or other. The components of the kit may also be provided in dried or lyophilized forms. When reagents or components are provided as a dried form, reconstitution generally is by the addition of a suitable solvent. It is envisioned that the solvent also may be provided in another container means.

The kit and components thereof may be packed for single use or for repeated usage, and the elements therein may be disposable such as to be disposed of after a single use or may be of a quality that allows repeated usage.

In addition to a diagnostic kit, the invention also provides a kit for use in therapeutic settings. In one embodiment, the kit comprises chitinase 3-like 1/Brp-39/YKL-40 or an activator thereof and an instructional material which describes, for instance, administering the chitinase 3-like 1/Brp-39/YKL-40 or an activator thereof to a subject as a prophylactic or therapeutic treatment or a non-treatment use as described elsewhere herein. In an embodiment, this kit further comprises a (preferably sterile) pharmaceutically acceptable carrier suitable for dissolving or suspending the therapeutic composition, comprising chitinase 3-like 1/Brp-39/YKL-40 or an activator thereof of the invention, for instance, prior to administering the molecule to a subject. Optionally, the kit comprises an applicator for administering the composition.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Human Validation of Chitinase-3-Like Protein 1 as a Biomarker of Recovery from Kidney Injury

While most studies of AKI have focused on the initiation of injury and its associated risk-factors and outcomes, there are far fewer studies that address the biology and clinical patterns of recovery. The experiments described herein were designed to identify factors that might promote the repair phase after kidney injury. A proteomic analysis of mouse urine after ischemia/reperfusion (I/R) injury was performed. Of the factors identified that were most highly upregulated in urine during kidney repair, several were fragments of the chitinase-like family of secreted proteins (CLPs).

The kidney tubule serves as a sentinel for organ ischemia because of the energy requirement imposed by high transport demands and the relatively hypoxicenvironment that accompanies the counter-current concentrating mechanism. Thus kidney hypoperfusion can lead to tubular cell death via necrosis and apoptosis, with corresponding loss of glomerular filtration and a subsequent rise in creatinine. These injurious stimuli trigger a series of responses that serve to limit the number of tubular cells that die and promote functional repair of the kidney. Using a urine proteomic screen in mice, the chitinase-like protein Brp-39 (the murine protein product of the chitinase 3-like 1 gene) was identified as a critical component of this reparative response that serves to limit tubular cell apoptotic death via activation of Akt and thus improve animal survival following kidney ischemia/reperfusion. Examination of graded times of renal ischemia revealed a direct correlation between the degree of kidney injury and the level of expression of Chi3l1/Brp-39 in the kidney and the urine. These findings were translated using urines collected from patients undergoing deceased donor kidney transplant, where it was shown that the orthologous human protein, YKL-40, was more highly expressed in urine from allografts that were subjected to sufficient peri-transplant ischemia to cause delayed graft function as opposed to those with slow or immediate graft function. Therefore, urinary levels of YKL-40 obtained within hours of transplant are highly predictive of the need for subsequent dialysis in these patients.

The materials and methods employed in these experiments are now described.

### Materials and Methods

Ischemia Reperfusion (I/R) Surgery and Experimental Protocol

All animal protocols have been approved by the Yale animal care and use committee. Nine to twelve week-old male WT and Brp-39−/− mice (on the C57BI/6 background, previously described by Lee et al. (2009, The Journal of Experimental Medicine 206: 1149-1166)) were anesthetized on a 37° C. warming pad, the abdomen opened, and warm renal ischemia induced using a non-traumatic microaneurysm clip (FST Micro Clamps, Foster City, Calif.) on the renal pedicle for the indicated time. To induce acute renal failure, the right kidney was surgically removed at the time of left kidney ischemia. During surgery the mice were hydrated with 1 ml of normal saline intraperitoneally (i.p.) and injected with 100 μl of Buprenex to avoid post-operative pain. The animals were additionally given 0.5 ml normal saline subcutaneously on day 1, and blood, urine and tissue samples obtained at the indicated times after I/R.

Histology and Immunocytochemistry

Kidneys were fixed in 4% paraformaldehyde (PFA) and embedded in paraffin. For histological evaluation of renal injury, sections were stained with hematoxylin and eosin and scored by the renal pathologist (GM), masked to the identity of the study animal. Multiple sections of renal tissue areas were evaluated for tubular necrosis, with or without regenerative features, and were scored using a square grid technique. Small squares of a 10×10 integrated grid, falling on tubules with morphologic features of overt necrosis (sloughing of cells, brush border loss, blebbing of cytoplasm) were counted in both cortex and outer medulla. Ten independent fields were counted per kidney (1000 squares per kidney), and the percentage of lesion area was calculated as percentage of total squares counted. To minimize the effect of injury variability on histological evaluation, kidney sections were scored from those animals that had BUN levels closest to the average BUN of all animals in that group.

For detection of proliferating cells, deparaffinized kidney sections were boiled in Retrievagen A buffer (BD Pharmingen, San Jose, Calif.), incubated overnight with rabbit anti-Ki-67 (1:50; Clone SP6; Thermo Fisher Scientific, Pittsburgh, Pa.), and visualized using Alexa 488 secondary antibody (Molecular Probes, Eugene, Carlsbad, Calif.). For detection of apoptotic cells, TUNEL positive nuclei were visualized using the In Situ Cell Death Detection Kit (Roche Diagnostics, Indianapolis, Ind.) as per the manufacturer's instructions. After labeling with TUNEL or Ki-67, tissue sections and cells were mounted in Vectashield DAPI and viewed with a Leica fluorescent microscope equipped with a 40×-Planapochromat objective and selective filters for fluorescein isothiocynate, DAPI and Texas red. Quantification of cells expressing the specified marker was performed in a blinded fashion by counting positive tubular cells/total tubular cells (identified as DAPI+ nuclei) in 10 randomly chosen 400× fields from the outer medulla.

FACS Analysis of Macrophage Populations

Kidneys were harvested, minced and a single cell suspension made by incubating with Liberase and DNAse-1 (Roche Diagnostics, Ind.) and filtered with a 40 μm cell strainer. Cells were stained with the following antibodies: Anti-F4/80 FITC-conjugated (eBiosciences, San Diego, Calif.); AntiCD45 PERCP-conjugated, Anti-Ly6C and Anti-CD11c (BD Biosciences, San Jose, Calif.), Antimannose receptor (AbD Serotec, Raleigh, N.C.) background set using the appropriate isotype controls. For quantification of total macrophage numbers/kidney, cell suspensions were mixed with a known amount of 5.1 μm AccuCount particles beads (Spherotech, Lake Forest, Ill.) before aliquoting and staining.

Cell Culture Experiments

Isolation of mouse proximal tubular epithelial cells (PTEC) was performed following a modified protocol by Schafer et al (Schafer et al., 1997, Am J Physiol 273:F650-657; Schmitt et al., 2008, J Am Soc Nephrol 19:2375-2383). Kidneys were harvested after cardiac perfusion with 0.025% collagenase (Worthington, Lakewood, N.J.) in M199 Hank's solution (Lonza, Walkersville, Md.). Renal cortex was isolated, minced, and then incubated at 37° C. in collagenase solution aerated with 5% CO₂ for 40 minutes. Tissue and cells were resuspended in renal epithelial basal medium (REBM, Lanza, Walkersville, Mo.) containing 2.5% FBS and penicillin/streptomycin, followed by passage through a 40 μm cell strainer. Cells were then plated and grown to confluence at 37° C. maintained in 5% CO₂ prior to incubation with recombinant Chi3L1/Brp-39 (RD Systems, Minneapolis, Minn.) and/or H₂O₂ (GIBCO, Langley, Okla.). MPT cells (Hader et al., 2010, Oncogene 29:1031-1040) were grown in DMEM/F12 media (GIBCO, Langley, Okla.) containing 10% serum and penicillin/streptomycin. For cell signaling experiments, cells were serum starved for 12 hours prior to incubation with recombinant Brp-39 (RD Systems, Minneapolis, Minn.) and protein extraction was performed. For in vitro induction of apoptosis, cells were incubated in 0.25 mM H₂O₂ for 6 hours±L Y294002 (50 μM, Promega, Madison, Wis.).

Immunoblot Analysis

Equal amounts of protein (30 μg) or mouse urine (40 μl) were loaded and electrophoresis was performed in a 10% polyacrylamide separating gel/5% stacking gel. Proteins were transferred to PVDF membrane, and blocked with 5% milk in TBST for 1 hour. The membrane was incubated over-night at 4° C. with primary antibodies: anti-phospho-Akt (S473, Cell Signaling, Danvers, Mass.), anti-phospho-p44/42 MAPK (Cell Signaling, Danvers, Mass.), anti-Brp-39 (generated as previously described by Lee et al. (2009, The Journal of Experimental Medicine 206: 1149-1166) and anti-Chi3L1 (RD Systems, Minneapolis, Minn.). Blots were washed in 0.1% TBST and incubated with secondary antibody for 35 minutes at room temperature. After washing, the second antibody was visualized by chemiluminescence reagents. Total AKT and total MAPK expression was determined using anti-Akt and anti-44/42 MAPK (Cell Signaling, Danvers, Mass.) as the loading control.

Analysis of mRNA/Real Time Polymerase Chain Reaction (qPCR)

RNA was extracted with a RNeasy Mini kit (Qiagen) and reverse transcribed. Gene expression analysis was determined by quantitative real-time PCR using an iCylcer iQ (Bio-Rad) and normalized to HPRT. Either a specific TaqMan Gene Expression Assay (Mm01545399_ml, Mm00801477_ml, Applied Biosystems, Foster City, Calif.) or the following primers were used with Cyber Green:

(SEQ ID NO: 1)
Chi3L1 Fw: CAAGGAACTGAATGCGGAAT;
(SEQ ID NO: 2)
Chi3L1 Rev: GGCTCCCAGACGTATCATGT;
(SEQ ID NO: 3)
HPRT Fw: CAGTACAGCCCCAAAATGGT;
(SEQ ID NO: 4)
HPRT Rev:CAAGGGCATATCCAACAACA.

Data are expressed using the comparative threshold cycle (dCt) method and mRNA ratios are given by 2-dCT.

Study Patients, Data Collection and Outcomes

This study was approved by the institutional review boards of all participating transplantation centers. Patients of at least 18 years old who were dialysis dependent and admitted to receive deceased donor kidney transplants were recruited. Patients who did not give informed, written consent or had primary non-function of the graft due to surgical complications were excluded. For further details, see prior publications from this cohort (Hall et al., 2011, Transplantation 91:48-56; Hall et al., 2010, J Am Soc Nephrol 21:189-197).

Experiments were designed to collect baseline donor, recipient and transplant characteristics consistent with the variables reported to UNOS (United Network for Organ Sharing). The need for dialysis, which was determined by the clinicians caring for participants at each institution without a standardized study protocol, was recorded by prospective chart review. DGF was defined as at least one dialysis session within seven days following transplant. In those without dialysis, SGF was defined as a creatinine reduction ratio (difference between the initial serum creatinine within an hour of transplant and the serum creatinine on day seven divided by the initial serum creatinine) less than 0.7, and IGF was defined as a creatinine reduction ratio greater than or equal to 0.7 (Johnston et al., 2006, Nephrol Dial Transplant 21:2270-2274).

Sample Selection

10 ml of urine and 6 ml of blood were collected upon arrival to the post-anesthesia care unit (time 0), typically within an hour of transplant. Urine samples at six, 12 and 18 hours after surgery, and on the first and second post-operative mornings (POD1 and POD2) were collected. Samples were centrifuged at 5000 g for 10 minutes to remove cellular debris and the supernatants were aliquoted into 1 ml samples for urine and 0.5 ml samples for blood. Samples were barcode labeled and stored at −80° C.

Statistical Analysis

Analyses were two-tailed with a significance level of 0.05. Chi-square or Fisher's exact tests were used to compare categorical variables. Analysis of variance (ANOVA) was used to compare mean values for continuous variables. Kruskal-Wallis tests were used to compare median values between those with OGF, SGF and IGF. Receiver-operating characteristic (ROC) curve analysis was performed to compare the accuracy of urinary and blood YKL-40 at each time point for predicting DGF.

The value for YKL-40 that gave the largest sum of sensitivity and specificity was chosen as the optimal cutoff SAS 9.2 for Windows (SAS Institute, Cary, N.C.) was used for all analyses.

The results of the experiments are now described.

### Brp-39 is Induced Following Ischemic Renal Injury

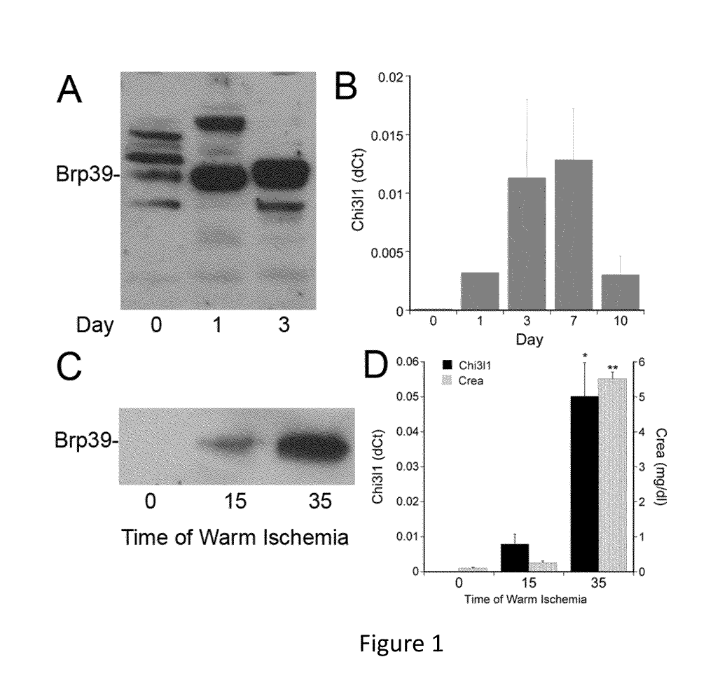

Mice were subjected to 25 minutes of bilateral renal I/R, and urine was collected at 1 and 3 days after injury and compared to urine from sham-operated mice. DIGE analysis revealed 11 peptides upregulated >2 times on day 3 after injury (at the time of peak tubule cell reparative proliferation) as compared to day 1 (time of peak injury) or sham. Of those identified by mass spectroscopy, 3 were fragments of chitinase 3-like proteins, suggesting that CLPs are upregulated in response to AKI. Western analysis with a-Brp-39 confirmed high expression of this CLP in mouse urine on day 1 and 3 after I/R as compared to baseline (FIG. 1A).

Quantitative RT-PCR of mRNA from mouse kidney outer medulla (OM) revealed nearly undetectable levels of Chi3l1 at baseline, with I/R inducing a >10 fold increase in gene expression peaking on days 3-7 after injury and returning to baseline by day 10 when repair is essentially complete (FIG. 1B). The level of Chi3l1/Brp-39 expression was found to correlate with the severity of ischemic injury. Fifteen minutes of warm ischemia, which causes minimal tubular injury and little loss of GFR, resulted in a modest upregulation of Chi3/1 mRNA in the kidney and Brp-39 protein in the urine, whereas 35 minutes of I/R, which leads to severe tubular necrosis and significant mortality, induced a more substantial increase in both kidney mRNA expression and urinary protein levels (FIG. 1C, 1D).

### Brp-39/Chi3L1 is Required for Normal Renal Responses to I/R Injury In Vivo

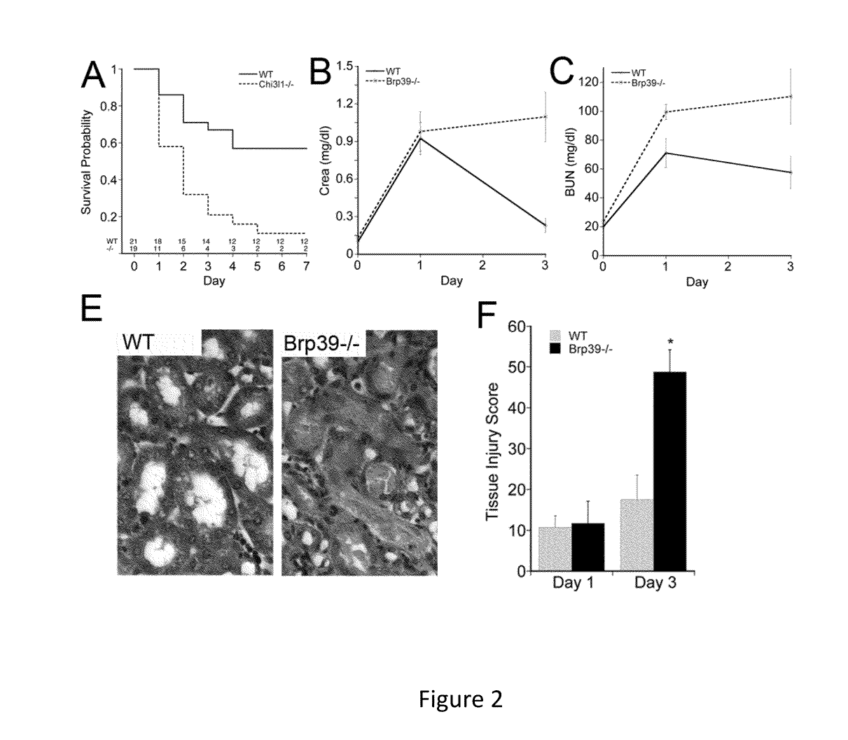

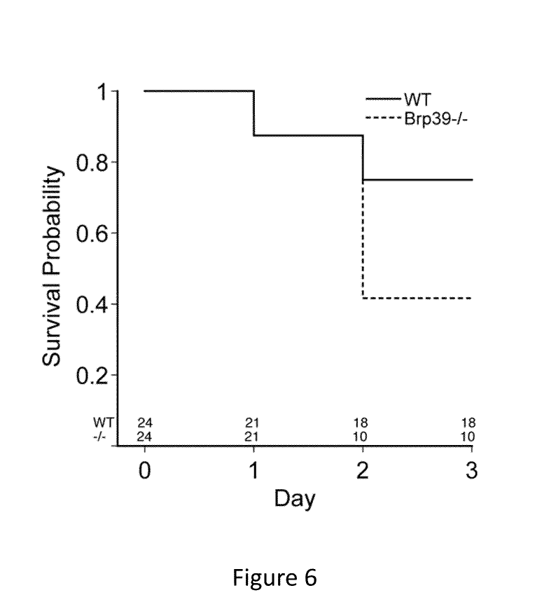

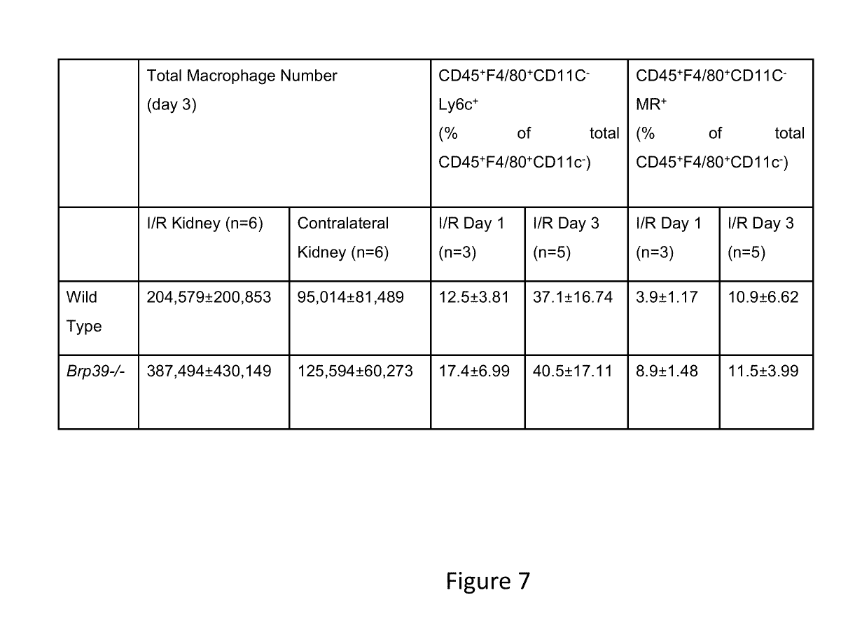

To determine the functional role of Brp-39 expression in AKI, wild-type and Brp-39^{-/-} mice were subjected to 30 minutes of unilateral warm ischemia and simultaneous contralateral nephrectomy. Mice lacking Brp-39 had a markedly increased mortality between 1 and 3 days after AKI as compared to WT mice subjected to the same ischemia time (FIG. 2A). Sham operation in Brp-39^{-/-} mice did not result in any mortality, suggesting that the mice were dying due to AKI rather than a post-operative complication such as sepsis. Reduction of the warm ischemia time to 25 minutes resulted in improved survival, although Brp-39^{-/-} mice continued to demonstrate increased mortality as compared to controls (FIG. 6). Serum analysis of mice subjected to 25 minutes of unilateral I/R with contralateral nephrectomy revealed that creatinine and BUN values peaked on day 1 in WT mice followed by improvement by day 3. In contrast, Brp-39^{-/-} mice exhibited a progressive rise in BUN and creatinine on day 3 (FIG. 2B, 2C), even though creatinine and BUN values from those mice that died prior to day 3 were excluded from this data set. Consistent with a failure in the normal onset of renal repair mechanisms between days 1 and 3, Brp-39^{-/-} mice exhibited more tubular cell loss and cast formation on day 3 after injury than on day 1, with a worse tubular injury score (FIG. 2D, 2E). Analysis of macrophages isolated from these kidneys revealed no statistical difference in the proportion of pro-inflammatory and reparative phenotypes as compared to wild-type mice, or in total macrophage numbers (FIG. 7).

### Brp-39/Chi3L1 Activates Tubular Epithelial Cell Akt and Reduces Apoptosis

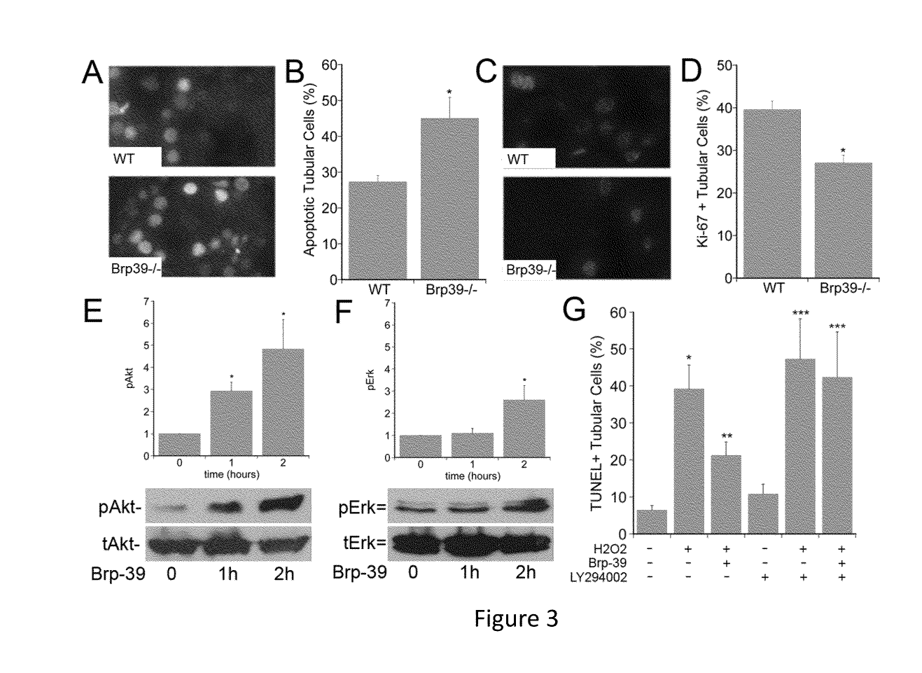

In light of the progressively worsening BUN values and tubular injury score seen in Brp-39^{-/-} mice after I/R, it was hypothesized that Brp-39 might function to moderate post-injury tubular cell apoptosis and/or activate subsequent regenerative responses in the ischemically injured kidney. Consistent with this, TUNEL staining performed on day 3 after I/R revealed that Brp-39^{-/-} mice have significantly higher rates of tubular cell apoptosis in the outer medulla as compared to WT animals (FIG. 3A, 3B), with a coincident reduction in reparative tubular cell proliferation (FIG. 3C, 3D).

In immune cells, Brp39 has been shown to have an anti-apoptotic effect via activation of intracellular signaling pathways downstream of the PI 3-kinase and MAPK (Lee et al., 2009, The Journal of Experimental Medicine 206: 1149-1166; Lee et al., 2011, Annu Rev Physiol 73:479-501). To determine if Brp-39 can activate these anti-apoptotic pathways in renal epithelial cells, cultured mouse proximal tubular cells (MPT) (Sheridan et al., 1993, Am J Physiol 265:F342-350; Karihaloo et al., 2001, J Biol Chem 276:9166-9173) were stimulated with recombinant Brp-39 followed by immunoblotting for the phosphorylated (activated) forms of Akt and Erk1/2 (FIG. 3 E, 3F). Akt was strongly activated at 60 and 120 minutes after Brp-39 addition, whereas Erk activation was more modest and not detected until the 2 hour time point.

These results demonstrate that Brp-39 is capable of activating intracellular anti-apoptotic signaling pathways. To determine whether Brp-39 can directly inhibit tubular cell apoptosis, primary cultures of renal tubular cells (PTEC) were isolated for in vitro analysis. TUNEL staining of these cells revealed that freshly isolated PTEC exhibited a modest level of baseline apoptosis that is markedly increased by exposure to reactive oxygen species (ROS) via addition of H₂O₂ (FIG. 3G). Pre-treatment with recombinant Brp-39 decreased H₂O₂-induced PTEC apoptosis by nearly 50%. Inhibition of PI3-kinase activation using LY294002 prevented the Brp-39 mediated anti-apoptotic effect, suggesting that activation of this pathway is critical for the protective effects of Brp-39 (FIG. 3G).

### Urinary YKL-40 Levels Predict Delayed Graft Function and Need for Dialysis Following Kidney Transplantation

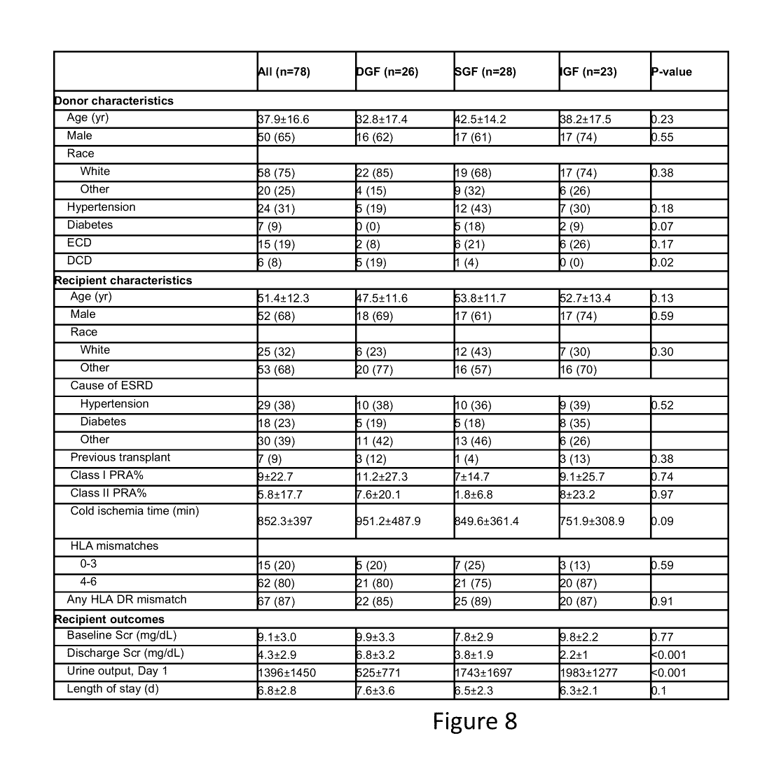

The observed correlation between the degree of ischemic renal injury and renal Chi3l1 mRNA expression in the mouse led to the consideration of the possibility that levels of urinary YKL-40, the protein produced by the human Chi3l1 gene, might be reflective of the degree of ischemic renal tubular injury in patients with AKI. To test this, urinary YKL-40 levels were compared in patients who received deceased-donor kidney transplants and exhibited delayed graft function (DGF indicative of severe ischemic injury (Muhlberger et al., 2009, Transplantation 88:S14-19)) to levels in those patients who had slow and immediate graft function (SGF and IGF, indicative of less severe ischemic injury). Urine and blood samples were collected from 78 patients at early time points after transplantation, of which 26 had DGF, 28 had SGF and 23 had IGF (Hall et al., 2011, Transplantation 91:48-56; Hall et al., 2010, J Am Soc Nephrol 21:189-197). Apart from a higher proportion of donations after cardiac death in the DGF group, donor and recipient characteristics were similar between the three groups (FIG. 8). As would be expected, mean urine output on the first post-operative day (POD) was lower and mean discharge serum creatinine was higher in the DGF group.

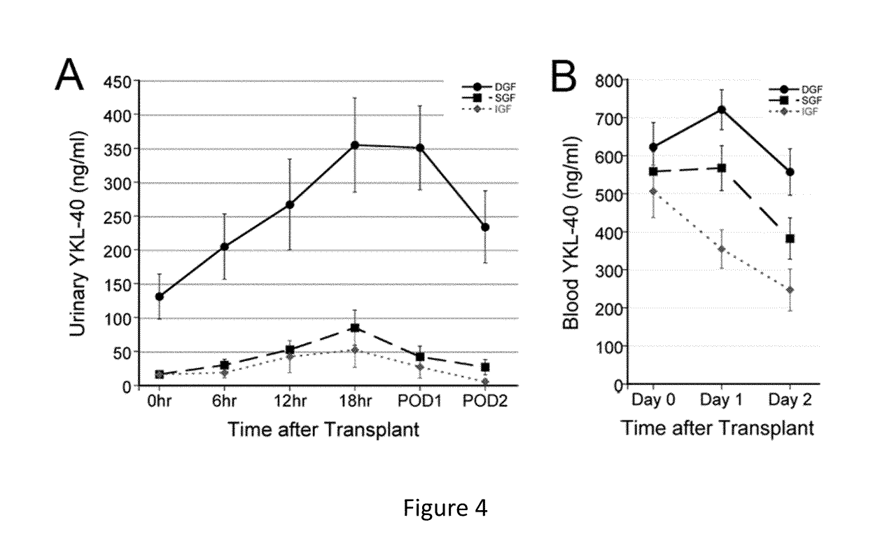

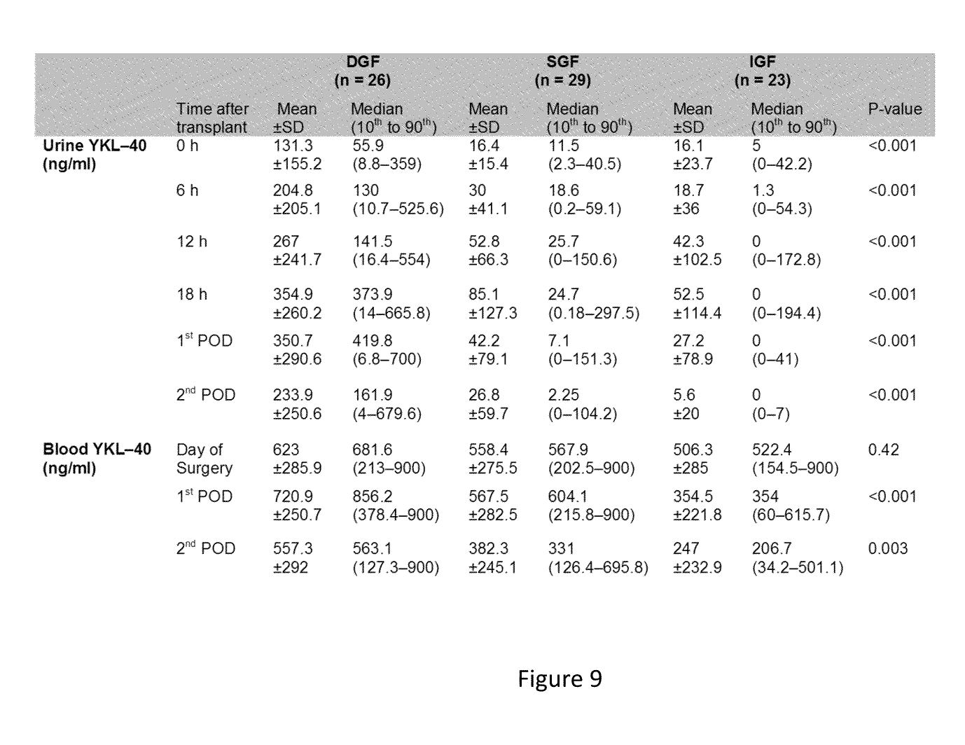

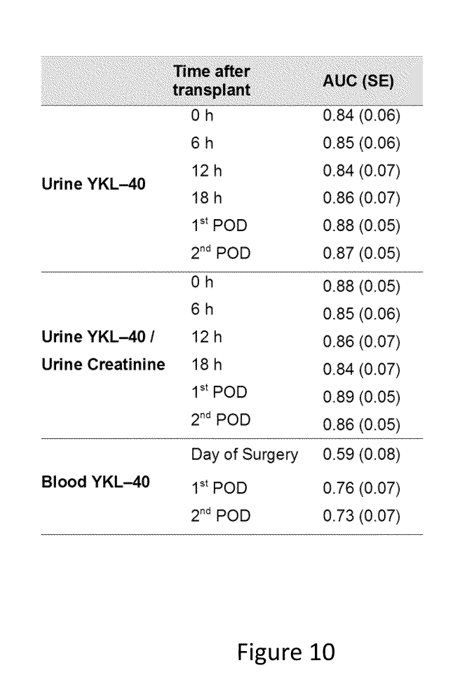

At all time points, mean urine YKL-40 values were highest in patients with DGF as compared to SGF and IGF (FIG. 4A). There was no statistically significant difference in blood YKL-40 values between groups immediately after surgery, but values separated significantly for both first and second post-operative days (FIG. 4B). FIG. 9 depicts mean and median values for both urine and blood YKL-40 measurements between groups at all time points. Normalization of urinary YKL-40 concentrations to urine creatinine did not significantly alter the differences observed between DGF patients and those not requiring dialysis (FIG. 10).

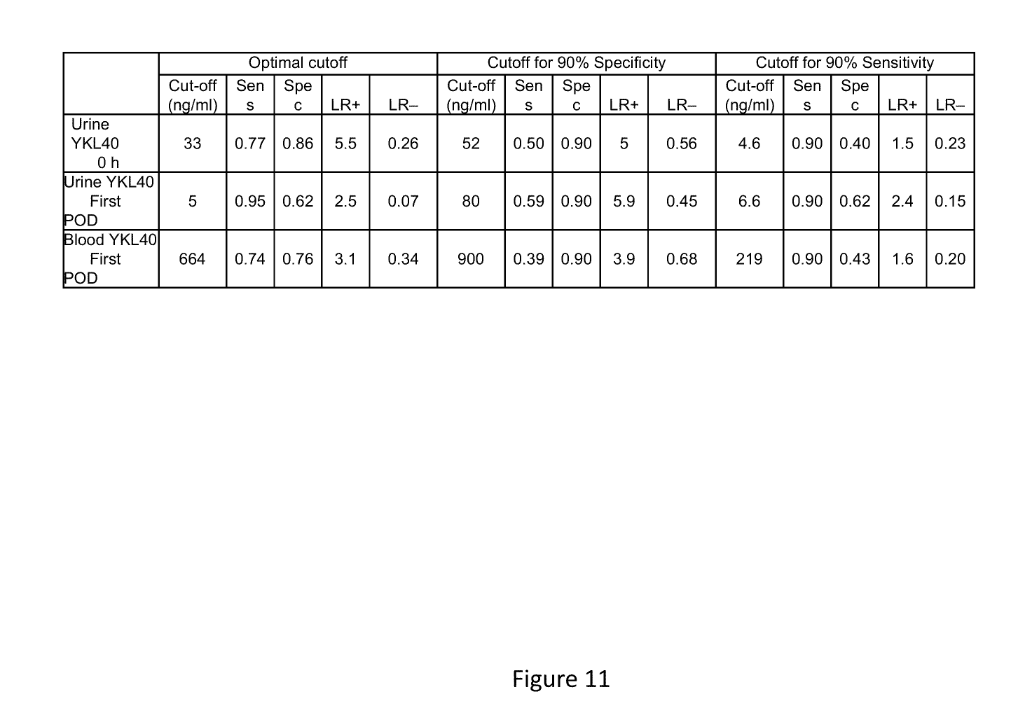

Receiver-operating characteristic curves indicated that urine YKL-40 predicted the development of DGF with moderately accurate areas under the curve AUGs (SE) of 0.84 (0.06) and 0.88 (0.05) at 0 hours and the first POD, respectively (FIG. 4). AUGs for blood YKL-40 at the same time points were 0.59 (0.08) and 0.76 (0.07). See FIG. 10 for AUGs for predicting DGF with urine and blood YKL-40 at all time points and FIG. 11 for the sensitivity and specificity of the biomarker at different cutoff values.

Other Biomarkers Analyzed in this Human Cohort

Experiments we performed to measure several urine biomarkers for predicting DGF at 0 hours and the first POD in this cohort (Hall et al., 2010, J Am Soc Nephrol 21:189-197; Hall et al., 2011, Am J Nephrol 33:407-413). In comparison with urine YKL-40, AUCs were 0.68 (0.07) and 0.82 (0.06) for neutrophil gelatinase-associated lipocalin (NGAL), 0.68 (0.06) and 0.82 (0.05) for interleukin-18 (IL-18), and 0.61 (0.07) and 0.50 (0.07) for kidney injury molecule-1 (KIM-1). The AUCs for previously measured blood biomarkers were 0.38 (0.07) and 0.54 (0.07) for NGAL and 0.49 (0.08) and 0.51 (0.08) for IL-1B, respectively (Hall et al., 2011, Transplantation 91:48-56).

### Chitinase 3-Like 1 Regulates the Renal Response to Ischemic Injury and Predicts Delayed Allograft Function

An unbiased approach using differential 20 gel electrophoresis (DIGE) followed by mass spectrometry was performed to discover proteins that are predominantly expressed in the urine during kidney repair to further understand the process of recovery from acute kidney injury. The results presented herein demonstrate that levels of urinary CLP are markedly increased after kidney injury, correlating with upregulated renal expression of the mRNA for Chi3l1 that peaks during the time of kidney repair. In addition, the level of renal Chi3l1 mRNA expression and urinary Brp-39 excretion directly correlate with the severity of kidney injury.

Multiple cell types are known to express Brp-39, including endothelial cells and inflammatory cells, both of which have been shown to have a major role in the response to ischemic kidney injury (Jang et al., 2009, J Mol Med 87:859-864). In addition, by RT-PCR and western analysis, it was observed that tubular cells themselves express easily detectable amounts of Chi3l1/Brp-39. Without wishing to be bound by any particular theory, it is believed that multiple cells may respond to kidney injury by upregulating the expression of this protein.

It is known that severe ischemic kidney injury causes initial tubular cell necrosis followed by a wave of tubular cell apoptosis that peaks at 24-48 hours after injury. This apoptotic response is driven in part by the local release of reactive oxygen species and results in significantly worse tubular injury and kidney function, followed by a marked increase in proliferation of surviving tubular cells that functionally reconstitute the tubule (Wu et al., 2007, J Clin Invest 117:2847-2859; Bonventre et al., 2003, J Am Soc Nephrol 14:2199-2210; Mizuno and Nakamura, 2005, Am J Pathol 166:1895-1905). Using Brp-39 null mice, it was discovered that the upregulation of Brp-39 in response to ischemic injury is critical in inhibiting tubular cell apoptosis in vivo, and that this pathway serves to limit the severity of tubular injury and maintain sufficient kidney function to keep the animal alive and promote proliferation of viable tubular cells to effect subsequent kidney repair. Furthermore, in vitro analysis of non-immortalized cells demonstrated that Brp-39 acts directly on tubular cells to activate PI3K/Akt signaling and inhibit ROS-mediated apoptosis.

As compared to the relatively simple animal model of renal artery clamping followed by reperfusion, patients are often exposed to ischemic kidney injury in the setting of multiple insults, making it more difficult to identify specific pathophysiologic events that are critical to the initial injury and subsequent repair. In those patients undergoing kidney transplantation, these factors include donor characteristics such as advanced age, donation after cardiac death (DCD) and cold ischemia time of the procured organ, as well as recipient characteristics such as body habitus, warm ischemia time and immunosuppressive regimens (Muhlberger et al., 2009, Transplantation 88:S14-19). By analyzing kidney biopsy specimens, Schwarz and coworkers found that tubular cells from patients with DGF had a significant increase in apoptotic responses with failure to upregulate typical anti-apoptotic pathways such as Bcl-2 and Bcl-xL (Schwarz et al., 2002, Lab Invest 82:941-948). Using a non-biased gene expression profiling approach, Mas and colleagues found a strong correlation between activation of the inflammatory response, particularly innate immunity markers such as IFITM1, BCL3, and C083, and the development of DGF in kidney transplant recipients (Mas et al., 2008, Transplantation 85:626-635).

While it is clear that immune activation can lead to apoptosis in injured organs, it has been demonstrate that the innate immune response also plays a critical role in the reparative events after injury (Ricardo et al., 2008, J Clin Invest 118:3522-3530; Jiang and Liao, 2010, J Cardiovasc Transl Res 3:410-416). For example, monocytes that enter the ischemically injured kidney adopt a pro-inflammatory expression profile in the first 24-48 hours, but then transition to an immune modulatory, pro-reparative phenotype during the following several days (Lee et al., 2011, J Am Soc Nephrol 22:317-326). In the experiments presented herein, it was demonstrated that YKL-40, known to be secreted by neutrophils and monocytes/macrophages as part of the innate immune response to injury, is markedly elevated in the urine of patients with DGF as compared to those with SGF or IGF, even though initial blood levels are indistinguishably elevated in all three groups. Based on the studies in ischemically injured mice, it is believed that the high urinary YKL-40 levels in patients with DGF come from the injured kidneys themselves and are indicative of greater tubular injury in those kidneys. Consistent with this, the group of patients who experienced DGF included a significantly higher number of recipients of kidneys from DCD donors and exhibited significantly lower urine outputs.

By combining studies of mice subjected to kidney I/R to identify the timing and importance of Brp-39 expression in the pathophysiology of I/R injury with studies in transplant recipients that establish a correlation between urinary YKL-40 and the severity of I/R injury during transplantation, additional experiments can be designed to further investigate this pathway. First, urinary expression of YKL-40 may provide a pre-procurement indicator of which kidneys will be the greatest risk for DGF. The fact that high levels of this protein are present in the urine immediately after transplantation suggests that many kidneys that exhibit DGF are likely to have suffered significant ischemic injury prior to or during organ procurement. I/R injury to such kidneys can be minimized via machine perfusion, with decreased risk for DGF and better one-year allograft survival (Moers et al., 2009, N Engl J Med 360:7-19). The modality is expensive however, and could be more cost-effective if reserved for the subgroup of allografts identified as being at highest risk for DGF based on determination of donor biomarkers such as urinary YKL-40. In addition, recent trials have demonstrated acceptable outcomes (with somewhat better long-term allograft function) for belatacept-based immunosuppressive regimens as compared to calcineurin inhibitor-based therapy (Ferguson et al., 2011, Am J Transplant 11:66-76; Vincenti et al., 2005, N Engl J Med 353:770-781). Trials designed to evaluate the efficacy of these and other calcineurin-sparing regimens can be planned in recipients identified as high-risk for DGF based on urinary YKL-40 levels before or immediately after transplant.

A second use of urinary YKL-40 may be as an indicator of the degree and duration of repair pathway activation that occurs following kidney transplantation. In the mouse model of moderate I/R injury, Chi3l1 levels peaked on days 3-7 and returned to baseline by day 10, paralleling the course of successful kidney repair. In patients with IGF or SGF, urinary YKL-40 levels were consistently low, suggesting that ischemic injury of these kidneys was mild. In contrast, those with DGF exhibited high urinary YKL-40 levels at all three time points, suggesting more severe I/R injury and marked activation of this reparative pathway. In fact, urinary YKL-40 levels immediately after transplant were far more accurate at predicting subsequent DGF than were urinary NGAL, IL-18 or KIM-1 levels. Work in rodent models of kidney injury has shown that repair pathways, including the innate immune response, are critical in re-establishing normal tubular function (Lee et al., 2011, J Am Soc Nephrol 22:317-326; Lin et al., 2010, Proc Natl Acad Sci USA 107:4194-4199; Menke et al., 2009, J Clin Invest 119:2330-2342). However, sustained activation of these same pathways in more severely injured kidneys can lead to maladaptive attempts at repair with fibrosis and nephron loss rather than tubule regeneration (Ricardo et al., 2008, J Clin Invest 118:3522-3530; Carlos et al., 2010, Transplantation 89:1362-1370; Lin et al., 2009, J Immunol 183:6733-6743). In support of the possibility that this paradigm may hold true in transplant recipients, allografts that recover promptly have excellent short and long term outcomes compared to those with delayed recovery (Perico et al., 2004, Lancet 364: 1814-1827). In fact, the increasing use of extended criteria donor kidneys over the past decade has led to a greater risk for DGF and a concomitant plateau in long term allograft survival despite a decline in acute rejection rates during this time (Tang et al., 2007, Semin Nephrol 27:377-392; 2010, US. Renal Data System, USRDS 2010 Annual Data Report: Atlas of Chronic Kidney Disease and End-Stage Renal Disease in the United States. Bethesda, Md.: National Institutes of Health, National Institute of Diabetes and Digestive and Kidney Diseases). This suggests that the severe I/R injury that underlies DGF may permanently compromise allograft function and lead to prolonged attempts at unsuccessful repair. By providing a better understanding of the degree of injury and the extent and duration of the pathophysiologic response to that injury, biomarkers like YKL-40 have the potential to improve both early and late outcomes following kidney transplantation.

Of note, YKL-40 performed well as a biomarker for predicting DGF when measured in both urine and blood, while other structural biomarkers have not (NGAL, IL-18 and KIM-1). Without wishing to be bound by any particular theory, it is believed that serum YKL-40 levels may provide new insights into the systemic effects of chronic kidney disease. A recent study has demonstrated that normal subjects have plasma YKL-40 levels of approximately 40 ng/ml (Bojesen et al., 2011, Clin Chim Acta 412:709-712), whereas patients who are at increased risk for cardiovascular disease have significantly higher levels that are believed to reflect the chronic activation of macrophages in atherosclerotic plaques (Kjaergaard et al., 2010, Ann Neurol 68:672-680; Bilim et al., 2010, J Card Fail 16:873-879). In the patient population of the present study, the average blood level of YKL-40 was more than 500 ng/ml in all three patient groups when measured immediately after transplantation. Patients with SGF and IGF had a subsequent decline in blood YKL-40 levels whereas those requiring dialysis did not, resulting in statistically significant differences on the first and second PODs. The well-described correlation between the development of chronic kidney disease (CKD) and accelerated risk of cardiovascular complications suggest that CKD/ESRD patients may constantly exhibit high circulating levels of YKL-40 and that the rapid decline observed in those with IGF following transplantation represents early resolution of this inflammatory state.

Cumulatively, the data presented herein demonstrate an unexpected role of chitinase-like proteins in ischemic organ injury and reinforce the concept that the innate immune response has evolved to identify and dispose of cells that are severely injured while promoting the survival and expansion of sublethally injured cells to effect subsequent organ repair. The discovery of chitinase 3-like 1/Brp-39/YKL-40 as both a sensor of the degree of injury and a critical mediator of this reparative response provides a potentially powerful biomarker that can promote rapid identification of those patients at greatest risk to have sustained renal failure following transplantation. It is believed that chitinase 3-like 1/Brp-39/YKL-40 may also predict the severity of injury in native kidneys and/or in other organ systems.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-9. (canceled)

10. A method of predicting recovery from an acute kidney injury in a mammalian subject, the method comprising the steps of:
a) measuring the level of YKL-40 present in a first body sample obtained from a first subject at a first time point;
b) measuring the level of YKL-40 present in a second body sample obtained from the subject at a later time point;
c) comparing the level of YKL-40 in the first body sample to the level of YKL-40 present in the second body sample;
wherein, when the level of YKL-40 is elevated in the second body sample compared to the level of YKL-40 present in the first body sample, the subject is predicted to be recovering from an acute kidney injury.

11. (canceled)

12. (canceled)

13. The method of claim 10, wherein the body sample is at least one body sample selected from the group consisting of a tissue, a cell, and a body fluid, further wherein said body fluid is urine.

14. (canceled)

15. The method of claim 10, wherein the measuring of the YKL-40 comprises an immunoassay for assessing the level of the YKL-40 in the sample.

16. The method of claim 15, wherein the immunoassay is at least one immunoassay selected from the group consisting of Western blot, ELISA, immunoprecipitation, immunohistochemistry, immunofluorescence, radioimmunoassay, dot blotting, and FACS.

17. The method of claim 10, wherein the measuring of the YKL-40 comprises a nucleic acid assay for assessing the level of a nucleic acid encoding the YKL-40 in the sample.

18. The method of claim 17, wherein the nucleic acid assay is at least one nucleic acid assay selected from the group consisting of a Northern blot, Southern blot, in situ hybridization, a PCR assay, an RT-PCR assay, a probe array, and a gene chip.

19. A method of predicting delayed graft function after kidney transplantation in a mammalian subject, the method comprising the steps of:
a) measuring the level of YKL-40 present in a first body sample obtained from a first subject at a first time point;
b) measuring the level of YKL-40 present in a second body sample obtained from the subject at a later time point;
c) comparing the level of YKL-40 in the first body sample to the level of YKL-40 present in the second body sample;
wherein, when the level of YKL-40 is elevated in the second body sample compared to the level of YKL-40 present in the first body sample, the delayed graft function after kidney transplantation is predicted in the subject.

20. (canceled)

21. (canceled)

22. The method of claim 19, wherein the body sample is selected from the group consisting of a tissue, a cell, and a body fluid, further wherein said body fluid is urine.

23. (canceled)

24. The method of claim 19, wherein the measuring of the YKL-40 comprises an immunoassay for assessing the level of the YKL-40 in the sample.

25. The method of claim 24, wherein the immunoassay is at least one immunoassay selected from the group consisting of Western blot, ELISA, immunoprecipitation, immunohistochemistry, immunofluorescence, radioimmunoassay, dot blotting, and FACS.

26. The method of claim 19, wherein the measuring of the YKL-40 comprises a nucleic acid assay for assessing the level of a nucleic acid encoding the YKL-40 in the sample.

27. The method of claim 26, wherein the nucleic acid assay is at least one nucleic acid assay selected from the group consisting of a Northern blot, Southern blot, in situ hybridization, a PCR assay, an RT-PCR assay, a probe array, and a gene chip.

28. A method of diagnosing for dialysis treatment in a mammalian subject following kidney transplantation, the method comprising the steps of:
a) measuring the level of YKL-40 present in a first body sample obtained from a first subject at a first time point;
b) measuring the level of YKL-40 present in a second body sample obtained from the subject at a later time point;
c) comparing the level of YKL-40 in the first body sample to the level of YKL-40 present in the second body sample;
wherein, when the level of YKL-40 is elevated in the second body sample compared to the level of YKL-40 present in the first body sample, the subject is diagnosed for needing dialysis treatment.

29. (canceled)

30. (canceled)

31. The method of claim 28, wherein the body sample is selected from the group consisting of a tissue, a cell, and a body fluid, further wherein said body fluid is urine.

32. (canceled)

33. The method of claim 28, wherein the measuring of the YKL-40 comprises an immunoassay for assessing the level of the YKL-40 in the sample.

34. The method of claim 33, wherein the immunoassay is at least one immunoassay selected from the group consisting of Western blot, ELISA, immunoprecipitation, immunohistochemistry, immunofluorescence, radioimmunoassay, dot blotting, and FACS.

35. The method of claim 28, wherein the measuring of the YKL-40 comprises a nucleic acid assay for assessing the level of a nucleic acid encoding the YKL-40 in the sample.

36. The method of claim 35, wherein the nucleic acid assay is at least one nucleic acid assay selected from the group consisting of a Northern blot, Southern blot, in situ hybridization, a PCR assay, an RT-PCR assay, a probe array, and a gene chip.

37. A method of treating a mammalian subject diagnosed with a kidney injury, the method comprising administering to the subject a composition comprising a therapeutically effective amount of YKL-40 or an activator of YKL-40 wherein the composition attenuates, prevents, or halts kidney cell apoptosis.

38. (canceled)


SEQUENCE LISTING (4 sequences)

SEQ ID NO: 1 - DNA, 20 bp, Artificial sequence
  Chemically synthesized
caaggaactgaatgcggaat

SEQ ID NO: 2 - DNA, 20 bp, Artificial sequence
  Chemically synthesized
ggctcccagacgtatcatgt

SEQ ID NO: 3 - DNA, 20 bp, Artificial sequence
  Chemically synthesized
cagtacagccccaaaatggt

SEQ ID NO: 4 - DNA, 20 bp, Artificial sequence
  Chemically synthesized
caagggcatatccaacaaca